(19) United States
(12) Patent Application Publication
(10) Pub. No.: US 2023/0256095 A1
(43) Pub. Date: Aug. 17, 2023

(54) DYES FOR USE IN A METHOD OF TREATMENT OF VITREOUS OPACITY-RELATED DISEASES

(71) Applicant: UNIVERSITEIT GENT, Gent (BE)

(72) Inventors: Félix SAUVAGE, Roubaix (FR); Stefaan DE SMEDT, Mariakerke (BE); Kevin BRAECKMANS, Daknam (BE); Katrien REMAUT, Gent (BE); Aranit HARIZAJ, Gent (BE)

(73) Assignee: UNIVERSITEIT GENT, Gent (BE)

(21) Appl. No.: 18/014,212

(22) PCT Filed: Jul. 9, 2021
(86) PCT No.: PCT/EP2021/069110

§ 371 Date: Jan. 3, 2023

(30) Foreign Application Priority Data
Jul. 10, 2020 (EP) 20185159.9

(51) Int. Cl. A61K 41/00; A61P 27/02; A61K 47/64; A61F 9/008; A61K 47/69

(52) U.S. Cl. CPC A61K 41/0038; A61P 27/02; A61K 47/643; A61F 9/00825; A61K 47/6935; A61F 2009/00874

(57) ABSTRACT

The invention concerns a dye for use in a method of treating a vitreous opacity-related disease in a subject. The method preferably comprises administering the dye to the vitreous body of an affected eye of the subject; and irradiating at least part of the vitreous opacity, thereby inducing destruction of the vitreous opacity in the subject. The invention further relates to the use of a dye for photodestruction of a vitreous opacity in an eye of a subject, and to a method of photodestruction of a vitreous opacity in an eye of a subject, the method comprising: administering a dye to the vitreous body of the eye of the subject; and irradiating at least part of the vitreous opacity, thereby inducing destruction of the vitreous opacity of the subject.

### FIELD OF THE INVENTION

The invention is broadly in the field of medicine, more precisely in the field of ophthalmology. In particular, the invention concerns the use of a dye in a method of treatment of a vitreous opacity-related disease in a subject.

### BACKGROUND OF THE INVENTION

The vitreous or vitreous body in the eye is a transparent gel mostly composed of water (99%) and formed by a network consisting of collagen and glycosaminoglycans such as hyaluronic acid (HA). With specific diseases such as myopia or diabetes and with aging, the vitreous liquefies, which, in some cases, can lead to post vitreous detachment and the formation of vitreous opacities. Those vitreous opacities which scatter light on the retina are responsible for vision degrading myodesopsia, which consists of the perception of floaters of various size and shape. Although floaters (or muscae volitantes in Latin) are not considered as an emergency in ophthalmology, some patients experiencing symptomatic floaters often complain from a loss in visual acuity and a strong negative impact on their quality of life.

There are currently not a large number of therapeutic solutions to treat eye floaters. The most used strategy is based on patient reassurance on how to live and cope with symptoms. Patients presenting highly symptomatic floaters often turn to non-conventional therapies because of a lack of therapeutic and medical options. Currently, there are two main therapeutic options for patients suffering from vitreous opacities. Based on the patient's eligibility, pars plana vitrectomy (PPV) is performed (i.e., the replacement of the vitreous by a saline solution or a gas). The second strategy relies on the use of the neodymium:yttrium aluminium garnet laser (Nd:YAG) with limited efficacy. Only about 30% of treated patients have seen improvements with this therapy. Even if most of the time those two strategies are safe, side effects such as cataracts or endophthalmitis can be associated.

In 2002, a retrospective study indicated 38% of patients treated with YAG laser found a moderate improvement in their symptoms against a full resolution of symptoms in 93.3% of eyes treated with PPV. However, surgical interventions like PPV can be associated to complications such as cataracts, retinal tears or endophthalmitis.

In a previous work, the efficacy of cationic gold nanoparticles and hyaluronic acid-coated gold nanoparticles (HA-AuNPs) was investigated to destroy artificial and human vitreous opacities.

Cationic gold nanoparticles became immobilized at the injection spot and were not able to reach the vitreous opacities. Small HA-AuNPs (10 nm) were able to bind and generate vapour nanobubbles (VNBs) on the opacities when illuminated with a nanosecond laser leading to their destruction (Sauvage et al., 2019, ACS Nano., 13, 8401-8416). However, due to concerns on the toxicity of gold nanoparticles—especially due the fact that they are non-biodegradable and fragment after laser illumination—there remains a need in the art for further and/or improved treatment options for the ablation of vitreous opacities and the concomitant treatment of vitreous opacity-related diseases.

### SUMMARY OF THE INVENTION

The present inventors have found compounds for use in a method of treating a vitreous opacity-related disease, thereby addressing one or more of the above-mentioned problems in the art.

Accordingly, a first aspect of the invention relates to a dye for use in a method of treating a vitreous opacity-related disease in a subject.

Preferably, the invention provides a dye for use in a method of treating a vitreous opacity-related disease in a subject, wherein the method comprises:
administering the dye to the vitreous body of an affected eye of the subject; and
irradiating at least part of the vitreous opacity, thereby inducing destruction of the vitreous opacity in the subject.

As shown in the experimental section, the present inventors have found that dyes such as Trypan Blue or Indocyanine Green are capable of diffusing in the vitreous body and accumulating at a vitreous opacity after administration, and can generate vapour nanobubbles (VNBs) at the vitreous opacity when irradiated. As shown in the examples, the so-obtained VNBs can provide sufficient mechanical forces to destroy vitreous opacities. Moreover, the VNBs were observed at the vitreous opacity only and not in its surroundings, thereby illustrating the targeted effect. Hence, the use of dyes advantageously avoids damage to the vitreous body and the ocular tissues surrounding the vitreous opacity.

As a further advantage, the dyes for use in the present methods allow destruction of the vitreous opacities when used at concentrations which are below the concentrations currently used in clinics. Besides, at those concentrations, no clear toxicity on retinal cells could be observed in vitro and in vivo. Moreover, the present treatment resulted in a clear destruction of both type I collagen fibers and opacities from patients with a strongly decreased number of pulses and laser energy (intensity) than current (YAG) laser therapy, thereby limiting the side effects on other tissues such as the retina. Furthermore, the dyes are biodegradable and approved for clinical use, in particular for ophthalmological use.

A further aspect relates to a method of photodestruction of a vitreous opacity in an eye of a subject, the method comprising:
administering a dye to the vitreous body of the eye of the subject; and
irradiating at least part of the vitreous opacity, thereby inducing destruction of the vitreous opacity of the subject.

A further aspect relates to the use of a dye for photodestruction (photoablation) of a vitreous opacity in an eye of a subject.

The above and further aspects and preferred embodiments of the invention are described in the following sections and in the appended claims. The subject-matter of appended claims is hereby specifically incorporated in this specification.

### DESCRIPTION OF THE DRAWINGS

FIG. 1: A: Graphs illustrating the size measurements (left) and zeta potential (right) of Indocyanine Green (ICG)-loaded nanoparticles; B: MTT assay of Müller cells (MIO-M1) treated with free and ICG-loaded nanoparticles for 24 hours (0.1-1 mg/ml); C: cell titer glo assay of Müller cells treated with different concentrations of trypan blue for 24 h (0.001-1 mg/ml). PAH: poly(allylamine) hydrochloride, HSA: human serum albumin, LIP-ICG: Liposomes encapsulating ICG.

FIG. 2: A: Dark field microscopy images of type I collagen fibers mixed with free ICG, different types of ICG nanoparticles (0.5 mg/ml), and TB (0.01 mg/ml), before and after illumination with a nanosecond laser (4.5 j/cm²; λ=561 nm). Dotted rectangles represent the position of the targeted fiber. The dotted circle represents the position of the laser beam. B: The average number of laser pulses required to destroy a type I collagen fiber with the nanosecond laser (4.5 j/cm²). For this experiment, fibers with an average diameter ranging from 500-1000 μm were chosen. C: Dark field image of a type I collagen fiber during one laser pulse (4.5 j/cm²). Bright spots that correspond to VNBs could be observed on the fiber but not outside. Scale bar=100 μm. PAH: poly(allylamine) hydrochloride, HSA: human serum albumin, LIP-ICG: Liposomes encapsulating ICG.

FIG. 3: A: Dark field images of free TB, free ICG and nano-encapsulated ICG in water before and during a laser pulse at a fluence of 4.5 j/cm². B: Number of bubbles generated by TB, ICG and nano-encapsulated ICG as a function of laser fluence. PAH: poly(allylamine) hydrochloride, HSA: human serum albumin, LIP-ICG: Liposomes encapsulating ICG, TB: Trypan Blue.

FIG. 4: Dark field images of a human vitreous opacities treated with free ICG (0.5 mg/ml) at 561 nm (4.5 j/cm²) and 800 nm (1.1 j/cm²). Rectangles indicate the targeted opacity. Dotted circles indicate the position of the laser beam.

FIG. 5: Schematic representation of mixing collagen fibers with free ICG (1.25 mg/ml in water) prior to intravitreal injection of the ICG-fibers in the eyes of rabbits (upper panel). Colour fundus photograph, photoacoustic microscopy (PAM) images, and merged photograph of the intravitreal injected ICG-labelled fibers illustrating that these fibers could be imaged by PAM at 578 nm and 800 nm (lower panel).

FIG. 6: Schematic representation intravitreal injection of non-labelled collagen fibers and, 5 days later, ICG (1.25 mg/ml) (upper panel). 3D photoacoustic microscopy (PAM) images at 578 nm and 800 nm, and overlay image of the intravitreal injected fibers illustrating that ICG co-localized with the fibers (lower panel).

FIG. 7: Schematic representation of intravitreal injection of collagen fibers in the eyes of rabbits, followed after 5 days by intravitreal injection of ICG (day 0) and laser treatment (day 3).

FIG. 8: Photographs illustrating that intravitreally injected ICG diffuses away from the injection spot and progressively gets cleared from the vitreous. Concentration of ICG: A: 1.25 mg/ml; B: 0.625 mg/ml; C: 0.25 mg/ml. After 7 days, ICG remained only detectable at the level of the injected collagen fiber, if a concentration of ICG of 1.25 mg/ml or 0.625 mg/ml was used.

FIG. 9: 2D optical coherence tomography (OCT) images of an injected collagen fiber (indicated by a white arrow) without the injection of ICG, before and after treatment with a pulsed laser (5 scans; <7 ns; 800 nm; 1.9 j/cm²). A region of interest (4.5×4.5 mm², containing the injected fibers) was scanned with the laser (white dotted rectangle).

FIG. 10: 2D optical coherence tomography (OCT) images of an injected collagen fiber (indicated by a white arrow) followed by ICG injection at a concentration of A: 1.25 mg/ml and B: 0.625 mg/mL before and after treatment with a pulsed laser (5 scans; <7 ns; 800 nm; 1.9 j/cm²) illustrating the destruction of collagen fibers (5 scans). A region of interest (4.5×4.5 mm², containing the injected fibers) was scanned with the laser (white dotted rectangle).

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the term “one or more”, such as one or more members of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All documents cited in the present specification are hereby incorporated by reference in their entirety.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

By extensive experiment testing, the present inventors have found that dyes are excellent compounds to aid in the targeted destruction of vitreous opacities. Dyes, such as Trypan Blue and Indocyanine Green, were found not to immobilize at the injection spot but to be sufficiently mobile in the vitreous body to be able to reach the vitreous opacity fibers. This finding was unexpected in light of the presence of collagen in the vitreous body, and the known capability of dyes to stain the eye's inner limiting membrane composed of an intertwined network of collagen.

Accordingly, a first aspect of the invention relates to a dye for use in a method of treating a vitreous opacity-related disease in a subject.

Related aspects provide:
a method of treating a vitreous opacity-related disease in a subject in need of such a treatment, comprising administering a therapeutically effective amount of a dye to the subject.
the use of a dye for the manufacture of a medicament for the treatment of a vitreous opacity-related disease in a subject.
the use of a dye for the treatment of a vitreous opacity-related disease in a subject.

The terms “dye” or “stain” as used herein refer to a chemical compound that is capable of binding to various substances in nature to induce colour. Thereby, the dye may increase the visibility of the substance.

As shown in the example section, a dye such as indocyanine green advantageously binds to a vitreous opacity, thereby allowing accumulation of the dye at the vitreous opacity, and functions as a light absorbing agent, thereby allowing the localised destruction of the vitreous opacity.

In embodiments of the uses and methods as taught herein, the dye is capable of diffusing in the vitreous body. In embodiments of the uses and methods as taught herein, the dye is capable of binding to the vitreous opacity. In embodiments of the uses and methods as taught herein, the dye is capable of accumulating at the vitreous opacity.

In embodiments, the dye is a light absorbing or light sensitizing agent. In embodiments, the dye is capable of absorbing light, such as in the visible light range or near infrared range. Thus, the laser radiation used in embodiments of the methods as taught herein may be performed by using a laser emitting laser light in the visible spectrum, e.g. a wavelength of 561 nm. This advantageously makes the laser radiation used in the methods as taught herein visible to the clinician, e.g. as opposed to prior art (Nd:YAG) laser treatment to treat eye floaters that operates at 1064 nm, outside the visible spectrum. The radiation used in embodiments of the methods as taught herein may be performed by using a laser emitting laser light in the near infrared spectrum, e.g. a wavelength of 800 nm. This advantageously lowers interference with surrounding tissues and hence reduces side effects.

Accordingly, an aspect provides a dye for use in a method of treating a vitreous opacity-related disease in a subject as a light sensitizing agent.

In embodiments of the uses and methods as taught herein, the dye may be capable of forming vapour nanobubbles at the treating a vitreous opacity-related disease in a subject when irradiated.

The reference to “a dye” encompasses one or more dyes, such as two or more, three or more, or four or more, such as five, six, seven, eight or more dyes.

The reference to “a dye” encompasses salts thereof, such as pharmaceutically acceptable salts thereof.

A dye may be used on living cells that have been removed from an organism, or may be introduced into the body, e.g. by injection.

In embodiments of the uses and methods as taught herein, the dye may be a biocompatible dye.

In embodiments of the uses and methods as taught herein, the dye may be a vital dye.

The term “vital dye” generally refers to a dye that is capable of binding to living cells or components thereof (such as the vitreous opacity) without inducing immediate evident degenerative changes to the cells or components thereof.

A vital dye may be used on living cells that have been removed from an organism, or may be introduced into the body, e.g. by injection.

In embodiments, the dye such as the vital dye may be a natural dye or a synthetic dye.

In embodiments of the uses and methods as taught herein, the dye such as the vital dye may be a dye approved for ophthalmological use. In embodiments, the dye may be a vital dye approved for ophthalmological use.

In embodiments of the uses and methods as taught herein, the dye such as the vital dye may be a fluorescent dye. Such fluorescent dyes (e.g. ICG, fluorescein) allow further imaging/visualization of the vitreous opacity.

In embodiments of the uses and methods as taught herein, the dye such as the vital dye may be an amphiphilic dye. Such dyes advantageously diffuse through the vitreous and specifically bind to (accumulate at) vitreous opacities in the eye of a subject.

The term “amphiphilic” refers to the property of possessing both hydrophilic (water-loving, polar) and lipophilic (fat-loving) properties.

In embodiments of the uses and methods as taught herein, the dye such as the vital dye may be selected from the group consisting of an azo dye, an arylmethane dye, a cyanine dye, a thiazine dye, and a xanthene dye.

Examples of azo dyes include Trypan Blue (Membrane Blue, Vision Blue, CAS Number: 72-57-1) and Janus green B (Diazine Green S, Union Green B, CAS Number: 2869-83-2).

Examples of arylmethane dyes include Gentian violet (Crystal violet, Methyl violet 10B, Hexamethyl pararosaniline chloride, CAS Number: 548-62-9); Bromophenol Blue (CAS Number: 115-39-9); Patent blue (Blueron, CAS Number: 3536-49-0); Brilliant Blue (Acid Blue, Coomassie Brilliant Blue, Brilliant Peel, CAS Number: 6104-59-2); Light Green (Light Green SF, Light Green SF Yellowish, CAS Number: 5141-20-8); and Fast Green (Fast Green FCF, Food green 3, FD&C Green No. 3, Green 1724, Solid Green FCF, CAS Number: 2353-45-9).

Examples of cyanine dyes include Indocyanine Green (Cardiogreen, Foxgreen, Cardio-Green, Fox Green, IC Green, CAS Number: 3599-32-4) and Infracyanine Green. Infracyanine Green (IfCG) is a green dye with the same chemical formula and similar pharmacologic properties as ICG. IfCG dye possesses two pharmacologic differences when compared to ICG. First, IfCG contains no sodium iodine, which must be added to ICG during the dye synthesis. Second, the presence of the sodium iodine in the ICG solution necessitates dilution in water, resulting in a hypotonic solution.

Examples of thiazine dyes include Methylene blue (Methylthioninium chloride, CAS Number: 61-73-4) and Toluidine blue (CAS Number: 92-31-9).

Examples of xanthene dyes include Fluorescein Sodium (CAS Number: 518-47-8); Rose Bengal (CAS Number: 4159-77-7); and Rhodamine 6G (Rhodamine 590, Rh6G, C.I. Pigment Red 81, C.I. Pigment Red 169, Basic Rhodamine Yellow, C.I. 45160, CAS Number: 989-38-8).

In embodiments of the uses or methods as taught herein, the dye may be a vital dye selected from the group consisting of: Indocyanine Green (ICG), Trypan Blue (TB), Brilliant Blue (BB), Janus green B (JG), Gentian violet (GV), Bromophenol Blue (BPB), Patent blue (PB), Light Green (LG), Fast Green (FG), Infracyanine Green (IfCG), Methylene blue (MB), Toluidine blue (ToB), Fluorescein Sodium (FS), Rose Bengal (RB), and Rhodamine 6G (R6G). In embodiments, the dye may be Indocyanine Green, Trypan Blue, or Brilliant Blue. Such dyes are advantageously approved for use in ophthalmology.

Preferably, the dye is Indocyanine Green or Trypan Blue. Both ICG and TB allow destruction of the vitreous opacities when used at concentrations which are below their respective concentrations currently used in clinics. In addition, destruction can be obtained using ICG or TB with a strongly decreased number of pulses and laser energy (intensity) than existing laser therapy, thereby limiting the side effects on other tissues such as the retina.

More preferably, the dye is Indocyanine Green. Indocyanine Green advantageously has a wide range of absorbance. Therefore, ICG allows to tune the wavelength of the laser for instance to near infrared light, which advantageously lowers interference with surrounding tissues and hence reduces side effects.

The reference to “a vital dye” encompasses one or more vital dyes, such as two or more, three or more, or four or more, such as five, six, seven, eight or more vital dyes.

The reference to “a vital dye” encompasses salts thereof, such as pharmaceutically acceptable salts thereof.

In embodiments of the uses or methods as taught herein, the dye may be a free or unbound dye, an aggregate of the dye (e.g. H-aggregate or J-aggregate), or a crystal of the dye; or the dye (including an aggregate or crystal thereof) may be conjugated to an agent (e.g. a polymer, a lipid, a peptide, a protein) and/or the dye (including an aggregate or crystal thereof) may be comprised in a particle, such as a nanoparticle or a microparticle.

The dye such as the vital dye as taught herein can be a free dye or can be combined with or chemically bonded to other elements or compounds. In embodiments of the uses or methods as taught herein, the dye may be a free or unbound dye. Free dyes advantageously allow a localized effect at the vitreous opacity. Free dyes allow to specifically bind and efficiently destroy vitreous opacities, even at concentrations which are below the concentrations used in clinics. Without being bound to theory, this phenomenon is likely due to binding (accumulation) of the free dyes on the vitreous opacity which decreases the energy threshold for the generation of vapor nanobubbles.

The terms “free” or “unbound” denote that the dye is not combined with or chemically bonded to other elements or compounds, e.g. the dye is not conjugated to another agent, or the dye is not coupled (e.g. grafted) to or enclosed (e.g. encapsulated) in a particle. The free or unbound dyes as taught herein include but are not limited to dyes in solution, and dried or lyophilized dyes, such as a powder of dyes such as a lyophilized powder for injection.

In embodiments of the uses or methods as taught herein, the dye may be an aggregate of the dye (e.g. H-aggregate or J-aggregate) or a crystal of the dye. Such aggregates and crystals advantageously improve the destruction of vitreous opacities because aggregates and dyes remain longer in the vitreous. Due to their larger size, using aggregates or crystals of the dye reduces or avoids entering of the dye into the retina, hence limiting or avoiding retinal toxicity. Further, dye aggregation shifts the absorption wavelength up to higher wavelengths, such as further into the IR region (e.g. 800-900 nm), thereby reducing toxicity as tissues do not or only slightly absorb in this region. In addition, these higher wavelengths, such as in IR region (e.g. 800-900 nm), correspond to the wavelengths of the currently used lasers. Further advantages are the ease of synthesis of the aggregates and the fact they only have the dye in their structure (without any other agent such as polymer or lipid).

The free or unbound dye may be comprised in a composition or formulation such as a pharmaceutical formulation or kit of parts, as will be described further herein. The composition may comprise the dye in a concentration ranging from about 0.001 mg/ml to 5 mg/ml, such as in a concentration of 0.01 ml/ml to 1 mg/ml, or 0.1 mg/ml to 0.5 mg/ml.

Despite the fact that no toxicity was observed for free ICG and free TB at clinically used concentrations, it may be advantageous to conjugate the dye to an agent, or to comprise the dye in a particle, such as a nanoparticle or microparticle, in order to reduce or even avoid penetration of the dye in the retina. The inner limiting membrane covering the retina has pores which prevent crossing of compounds or particles e.g. with a size superior to 100 nm (Peynshaert et al., 2017, Drug Delivery, 24:1, 1384-1394). Hence, conjugating the dye to an agent and/or comprising the dye in a microparticle or nanoparticle reduces or avoids entering of the dye into the retina, hence limiting or avoiding retinal toxicity.

In embodiments of the uses or methods as taught herein, the dye (including aggregates or crystals thereof) may be conjugated to an agent (e.g. a polymer, a lipid, a peptide, a protein) and/or the dye (including aggregates or crystals thereof) may be comprised in a particle, such as a nanoparticle or a microparticle. In embodiments of the uses or methods as taught herein, the dye (including aggregates or crystals thereof) may be grafted on a particle and/or the dye (including aggregates or crystals thereof) may be encapsulated in a particle.

In embodiments of the uses or methods as taught herein, the dye such as the vital dye may be conjugated to an agent. The nature of the agent is not limiting, and the agent may be any chemical (e.g., inorganic or organic), biochemical or biological substance, molecule or macromolecule (e.g., biological macromolecule).

In embodiments, the agent may be a polymer, a lipid, a peptide, or a protein. Since the size of the conjugate is larger than the size of the dye itself, administering a conjugate advantageously avoids travel of the dye to other parts of the eye and hence reduces or even eliminates toxicity.

In embodiments, the polymer may be selected from the group consisting of hyaluronic acid (HA), poly(ethylene) glycol (PEG), poly(DL-lactic-co-glycolic acid) (PLGA), poly(lactic) acid (PLA), polycaprolactone, ethyl cellulose, cellulose acetophthalate, polylactic acid, cellulose, polyvinyl alcohol, polyethylene glycol, gelatine, collagen, silk, alginate, dextran, starch, polycarbonate, polyacrylate, polystyrene, poly(alkyl cyanoacrylate) (PACA), and polyoxazoline. Preferably, the polymer may be hyaluronic acid. For instance, the dye such as ICG may be conjugated to a polymer such as hyaluronic acid. Since the size of the ICG-HA conjugate is larger than the size of the dye itself, administering a conjugate advantageously avoids travel to other parts of the eye and hence reduces or even eliminates toxicity.

In embodiments, the lipid may be anionic, neutral, or cationic lipid. In embodiments, the lipid may be natural, synthetic or bacterial lipid.

Suitable examples of anionic lipids include phosphatidylserine (PS) and phosphatidylglycerol (PG).

Suitable examples of neutral lipids include prostaglandins, eicosanoids, glycerides, glycosylated diacyl glycerols, oxygenated fatty acids, very long chain fatty acids (VLCFA), palmitic acid esters of hydroxystearic acid (PAHSA), N-acylglycine (NAGly), and prenols.

Suitable examples of cationic lipids include multivalent cationic lipids; 1,2-di-O-octadecenyl-3-trimethylammonium propane (DOTMA); ethylphosphocholines (ethyl PC); dimethyldioctadecylammonium (DDAB); pH sensitive lipids; 1,2-dioleoyl-3-trimethylammonium propane (DOTAP); 3β-[N—(N′,N′-dimethylaminoethane)-carbamoyl]cholesterol (DC-Cholesterol); N4-Cholesteryl-spermine (GL67); and 1,2-dioleyloxy-3-dimethylaminopropane (DODMA).

Such lipids are commercially available from Avanti Polar Lipids (Alabama, USA). For instance, a suitable multivalent cationic lipid is (N1-[2-((1S)-1-[(3-aminopropyl)amino]-4-[di(3-amino-propyl)amino]butylcarboxamido)ethyl]-3,4-di[oleyloxy]-benzamide). Examples of ethyl PC include 1,2-dilauroyl-sn-glycero-3-ethylphosphocholine (chloride salt) (12:0 EPC Cl salt); 1,2-dimyristoyl-sn-glycero-3-ethylphosphocholine (chloride salt) (14:0 EPC Cl salt); 1,2-dipalmitoyl-sn-glycero-3-ethylphosphocholine (chloride salt) (16:0 EPC Cl salt); 1,2-distearoyl-sn-glycero-3-ethylphosphocholine (chloride salt) (18:0 EPC Cl salt); 1,2-dioleoyl-sn-glycero-3-ethylphosphocholine (chloride salt) (18:1 EPC Cl salt); 1-palmitoyl-2-oleoyl-sn-glycero-3-ethylphosphocholine (chloride salt) (16:0-18:1 EPC Cl salt); and 1,2-dimyristoleoyl-sn-glycero-3-ethylphosphocholine (Tf salt) (14:1 EPC Tf salt).

Examples of pH sensitive lipids include N-(4-carboxybenzyl)-N,N-dimethyl-2,3-bis(oleoyloxy)propan-1-aminium (DOBAQ); 1,2-distearoyl-3-dimethylammonium-propane (18:0 DAP); 1,2-dipalmitoyl-3-dimethylammonium-propane (16:0 DAP); 1,2-dimyristoyl-3-dimethylammonium-propane (14:0 DAP); 1,2-dioleoyl-3-dimethylammonium-propane (18:1 DAP or DODAP).

In embodiments of the uses and methods as taught herein, the dye may be comprised in a particle, such as a nanoparticle or a microparticle.

In embodiments, the particle may be a nanosphere or microsphere. The particle may also be a nanorod, a microrod, a nanostar, a microstar, a nanopyramid, a micropyramid, a nanoshell or a microshell. In embodiments, the particle may be a nanosphere. In embodiments, the particle may have a diameter in the range of 1 nm to 1000 nm, for instance 1 nm to 500 nm, e.g. in the range of 50 nm to 500 nm, preferably in the range of 100 nm to 400 nm, e.g. in the range of 150 nm to 300 nm. In embodiments, the particle may have a diameter in the range of 5 nm to 300 nm, for instance 10 nm to 250 nm, such as 150 nm to 250 nm.

It is an advantage of particle sizes, e.g. in the range of 100 nm to 300 nm, e.g. in the range of 150 nm to 250 nm, that the mobility of the particles in the vitreous may be improved.

In embodiments, the nanoparticles, for instance their core, may comprise a polymer material, carbon and/or titanium. The core may comprise melanin. The core may comprise poly-dihydroxyphenylalanine (DOPA).

The nanoparticle may be a polymer nanoparticle, a protein nanoparticle or lipid nanoparticle (i.e. liposome). The polymer may be poly(lactic-co-glycolic acid) (PLGA). For example, PLGA-based ICG nanoparticles (PLGA-ICG NPs) may be prepared as described in Saxena et al., 2004, Int J Pharm, 278(2):293-301. The protein may be human serum albumin (HSA). For example, human serum albumin ICG nanoparticles (HSA-ICG NPs) may be prepared as described in Sheng et al., 2014, ACS Nano, 8(12):12310-22. The lipid nanoparticle may be MC3-based lipid nanoparticles (Patel et al., 2019, J. Control. Release, 303, 91-100). Liposomes encapsulating ICG (Lip-ICG) may be prepared as described in Lajunen et al., 2018, J. Control. Release 284, 213-223.

The particles, e.g. the particle size, may be characterized by dynamic light scattering (DLS), transmission electron microscopy (TEM), UV-vis spectroscopy, and/or electrodynamic modeling using Mie theory. The obtained concentration of the particles may be estimated using experimental extinction intensities at the maximum wavelength, and Mie theory calculations of the extinction cross section for spherical particles. The encapsulation efficiency (i.e. dye loading efficiency) may be determined based on a calibration curve of free dye obtained by UV-vis spectrometry or fluorescence. The zeta-potential may be measured by electrophoretic mobility.

In embodiments of the uses and methods as taught herein, the dye may be grafted on a particle, such as a nanoparticle or a microparticle. In embodiments, the dye may be grafted on a nanoparticle. For instance, the dye may be grafted on a particle by ‘click-chemistry’ at the surface of the particle (e.g. at the end of polymer chains such as at the distal end of poly(ethylene) glycol chains or hyaluronic acid chains). For instance, the grafting of a dye may occur at the end of PEG chains that are grafted on the particles.

In embodiments of the uses and methods as taught herein, the dye may be encapsulated in a particle, such as a nanoparticle or a microparticle. In embodiments, the dye may be encapsulated in a nanoparticle. In embodiments, the dye may be encapsulated in a particle by physical or chemical encapsulation. For instance, physical encapsulation of ICG in liposomes may be performed by adding ICG during the rehydration of the lipids. For HAS-ICG particles, the chemical encapsulation may be performed by reacting ICG with the disulphide bonds of HAS.

The terms “vitreous body”, “vitreous humour” or “the vitreous” can be used interchangeably herein and refer to a clear gel that fills the space between the lens and the retina of the eyeball of humans and other vertebrates. The vitreous body contains water (98-99% of its volume is water) and a network consisting of collagen and glycosaminoglycans such as hyaluronic acid (HA).

In youth, HA and collagen fibrils form a supramolecular network that maintains transparency and confers a gel state to the vitreous body. With aging, re-organization of the molecular components in the vitreous body alters vitreous structure inducing gel liquefaction (Synchysis senilis). This liquefaction may be accompanied by a collapse of the collagen network that could induce the formation of other collagen-based structures in the form of light scattering opacities responsible for the phenomenon of floaters or may lead to a posterior vitreous detachment (PVD), in which the vitreous membrane is released from the sensory retina. During this detachment, the shrinking vitreous can stimulate the retina mechanically, causing the patient to see random flashes across the visual field, sometimes referred to as “flashers”, a symptom more formally referred to as photopsia. The ultimate release of the vitreous around the optic nerve head sometimes makes a large floater appear, usually in the shape of a ring (“Weiss ring”).

The terms “vitreous opacity”, “floater”, “vitreous floater” or “muscae volitantes” can be used interchangeably and refer to a deposit within the eye's vitreous humour. The term “vitreous opacity” encompass any type of floater, such as floaters originating from the liquefaction of the vitreous body; floaters which may be caused by embryological remnants; or floaters which may be acquired due to aging, trauma, iatrogenic, ocular or systemic metabolic pathologies.

The majority of floaters are due to the degenerative changes of the vitreous in which the vitreous network is disrupted, e.g. by aggregates of collagen adhering to the vitreous framework in netlike masses that are disruptive of normal vision. A floater may be perceived as a linear structure with nodules, or a meshwork of linear structures, that appears to drift in front of the eye, caused by a shadow cast on the retina.

In embodiments, the vitreous opacity to be treated may have a length in the range of 0.5 mm to 5 mm, such as in the range of 1 mm to 4 mm or 2 mm to 3 mm. In embodiments, the vitreous opacities to be treated may be close to the retina or to the lens, e.g. at a distance in the range of 0 mm to 5 mm, such as at a distance in the range of 1 mm to 4 mm. For example, the floater may be present in the bursa premacularis.

The terms “vitreous opacity-related disease” refers to any disease or disorder related to the presence of a vitreous opacity in the eye of a subject.

In embodiments, the vitreous opacity-related disease may be myodesopsia or posterior vitreous detachment.

The terms “myodesopsia”, “myodaeopsia”, “myiodeopsia”, or “myiodesopsia” refer to the perception of floaters. The perception of a floater may be characterized by shadow-like vision artefacts.

The term “posterior vitreous detachment” refers to a condition of the eye in which the vitreous membrane separates from the retina. PVD may be characterized by one or more of symptoms selected from the group consisting of flashes of light (photopsia), a sudden dramatic increase in the number of floaters, and a ring of floaters or hairs just to the temporal side of the central vision.

The term “eye” as used herein has its meaning as ordinary in the art and refers to the organs of the visual system.

The terms “subject”, “individual” or “patient” can be used interchangeably herein, and typically and preferably denote humans, but may also encompass reference to non-human animals, preferably warm-blooded animals, even more preferably mammals, such as, e.g., non-human primates, rodents, canines, felines, equines, ovines, porcines, and the like. The term “non-human animals” includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In certain embodiments, the subject is a non-human mammal. In certain embodiments, the subject is a human subject. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species.

Suitable subjects may include without limitation subjects presenting to a physician for a screening for a vitreous opacity-related disease, subjects presenting to a physician with symptoms and signs indicative of a vitreous opacity-related disease, subjects diagnosed with a vitreous opacity-related disease, and subjects who have received an alternative (unsuccessful) treatment for a vitreous opacity-related disease.

In embodiments of the uses or methods as taught herein, the method may comprise:
administering the dye to the vitreous body of an affected eye of the subject; and
irradiating at least part of the vitreous opacity, thereby inducing destruction of the vitreous opacity in the subject.

In embodiments, the method comprises administering the dye such as the vital dye to the vitreous body of an affected eye of the subject.

In embodiments of the uses and methods as taught herein, the dye such as the vital dye may be administered at a concentration of about 0.001 mg/ml to about 5.0 mg/ml. In embodiments, the dye such as the vital dye may be administered at a concentration of about 0.01 mg/ml to about 1.0 mg/ml. In embodiments, the dye such as the vital dye may be administered at a concentration of about 0.1 mg/ml to about 0.5 mg/ml. In embodiments of the uses and methods as taught herein, the dye such as the vital dye may be administered at a concentration of about 0.001 mg/ml to about 0.5 mg/ml. Such concentration is equal to or lower than the concentration of a dye typically used in the clinic (e.g. typically used concentration in the clinic, in particular in ophthalmology, of for example TB is 0.6 mg/ml and of ICG is 1.25 mg/ml). The concentration advantageously allows treatment with no toxicity to the surrounding ocular tissues. The concentration is in a range that is clinically acceptable and/or routinely used.

In embodiments, when using ICG as the dye, the dye may be administered at a concentration of at least 0.01 mg/ml, such as at least 0.1 mg/ml, preferably at a concentration of at least 0.5 mg/ml.

For instance, when using ICG as the dye, the dye may be administered at a concentration of about 0.001 mg/ml to about 1.0 mg/ml, such as at a concentration of about 0.001 mg/ml to about 0.5 mg/ml, or at a concentration of about 0.01 mg/ml to about 0.5 mg/ml, preferably at a concentration of about 0.1 mg/ml to about 0.5 mg/ml. Such concentration is lower than the current clinically used concentration and allows treatment without any toxicity to the surrounding ocular tissues.

In embodiments of the uses and methods as taught herein, when using Trypan Blue as the dye, the dye may be administered at a concentration of at least 0.001 mg/ml, preferably at a concentration of at least 0.01 mg/ml. For instance, when using Trypan Blue as the dye, the dye may be administered at a concentration of about 0.001 mg/ml to about 0.5 mg/ml, such as at a concentration of about 0.001 mg/ml to about 0.1 mg/ml, preferably at a concentration of about 0.001 mg/ml to about 0.01 mg/ml. Such concentration is lower than the currently used concentrations in the clinic and allows treatment without any toxicity to the surrounding ocular tissues.

In embodiments of the uses and methods as taught herein, the dye such as the vital dye may be administered to the vitreous body by intravitreal administration. Intravitreal administration advantageously allows delivery of the dye such as the vital dye directly to the vitreous body. In embodiments, the dye such as the vital dye may be administered to the vitreous body by injection. In embodiments of the uses and methods as taught herein, the dye such as the vital dye may be administered to the vitreous body by intravitreal injection. Intravitreal injection allows delivery of the dye such as the vital dye directly to the vitreous body by a minimally invasive technique, thereby reducing the risks and pain for the patient and increasing the patient's well-being.

The term “intravitreal administration” as used herein refers to a process or procedure to place a medication (e.g. the dye or composition as taught herein) directly into the vitreous cavity which is filled with the vitreous body.

In embodiments of the uses and methods as taught herein, the dye such as the vital dye may be capable of diffusing in the vitreous body after administration, for instance by intravitreal injection. When using the dye such as the vital dye in accordance with embodiments of the invention, the dye may diffuse in the vitreous body after administration.

In embodiments of the uses and methods as taught herein, the dye such as the vital dye may be capable of binding to (accumulating at) the vitreous opacity after administration, for instance by intravitreal injection. When using the dye such as the vital dye in accordance with embodiments of the invention, the dye may bind to (accumulate at) the vitreous opacity after administration.

The binding of the dye to the vitreous opacity may be binding by a covalent binding or a non-covalent interaction.

In embodiments, the method may comprise administering the dye to the vitreous body of an affected eye of the subject, thereby inducing binding of the dye to (accumulation of the dye at) the vitreous opacity. In embodiments, the method may comprise administering the dye to the vitreous body of an affected eye of the subject, thereby inducing diffusion of the dye in the vitreous body and binding of the dye to (accumulation of the dye at) the vitreous opacity.

In embodiments of the uses and methods as taught herein, the dye such as the vital dye may be capable of forming vapor nanobubbles at the vitreous opacity when irradiated. When using the dye such as the vital dye in accordance with embodiments of the invention, the dye may form vapor nanobubbles at the vitreous opacity when irradiated.

In embodiments, the method may comprise irradiating the dye bound to at least part of the vitreous opacity, thereby inducing destruction of the vitreous opacity in the subject. In embodiments, the method may comprise irradiating the dye bound to at least part of the vitreous opacity, thereby forming vapour nanobubbles at the vitreous opacity and inducing destruction of the vitreous opacity in the subject.

The wording “inducing destruction of”, “causing destruction of” or “destroying” may be used interchangeably herein.

In embodiments, the method may comprise:
administering the dye to the vitreous body of an affected eye of the subject, thereby inducing binding of the dye to (accumulation of the dye at) the vitreous opacity; and
irradiating the dye bound to (accumulated at) at least part of the vitreous opacity, thereby inducing destruction of the vitreous opacity in the subject.

The dye such as the vital dye as taught herein may be used as a light sensitizing agent in a method for the treatment of a vitreous opacity-related disease.

The treatment may comprise injecting the dye such as the vital dye into the vitreous body of an eye of a human or animal subject. The treatment may comprise a laser ablation treatment after injection of the dye such as the vital dye into the vitreous body of an eye of a human or animal subject.

When using the dye such as the vital dye in accordance with embodiments of the invention, the dye may specifically bind to the vitreous opacity and may locally exert a mechanical force onto the vitreous opacity when irradiated by laser light in the laser ablation treatment.

When using the dye such as the vital dye in accordance with embodiments of the invention, the dye may form vapor nanobubbles in the vitreous when being irradiated, so as to exert a mechanical force onto the vitreous opacity.

When using the dye such as the vital dye in accordance with embodiments of the invention as a light sensitizing agent in a method for treatment of a vitreous opacity-related disease, the dye may cluster around a vitreous opacity to concentrate energy deposition by the laser ablation treatment near and/or in the vitreous opacity, such that a collapse of the vapor nanobubbles releases a mechanical force to dislodge and/or break apart the vitreous opacities.

In embodiments of the uses and methods as taught herein, the method may comprise irradiating at least part of the vitreous opacity, thereby inducing destruction of the vitreous opacity in the subject. In embodiments, the method may comprise irradiating at least part of the vitreous opacity with radiation to dislodge and/or break apart and/or destruct the vitreous opacity in the subject.

The phrase “destruction of the vitreous opacity” as used herein refers to fragmentation of the vitreous opacity.

In embodiments, the use of the dye such as the vital dye as taught herein may lead to fragmentation of a vitreous opacity in at least two fragments. For example, the use of the dye as taught herein may lead to fragmentation of a vitreous opacity in two or more fragments, such as five or more, ten or more, twenty or more, fifty or more, or hundred or more fragments. The size of the resulting fragments (i.e. fragments after the treatment) may be at most 50% of the size of the vitreous opacity before treatment. For example, the size of the resulting fragments (i.e. fragments after the treatment) may be at most 40%, at most 30%, at most 20%, at most 10%, at most 5%, at most 1%, at most 0.1%, or at most 0.01% of the size of the vitreous opacity before treatment. The resulting fragments may be visible or may no longer be visible after the treatment, for instance when observed by microscopic analysis.

When using a dye such as a vital dye as taught herein, it is possible, due to the dye's localized effect, to irradiate only part of the vitreous opacity to fragment the vitreous opacity.

In embodiments of the uses and methods as taught herein, the at least part of the vitreous opacity may be irradiated with electromagnetic radiation. In embodiments, the at least part of the vitreous opacity may be irradiated with laser radiation. In embodiments, the at least part of the vitreous opacity may be irradiated with pulsed-laser radiation.

The terms “radiation” and “electromagnetic radiation” may be used interchangeably herein.

In embodiments, the electromagnetic radiation is infrared radiation (including near infrared) or visible light.

Laser irradiation, such as irradiation by pulsed lasers, e.g. pico-, femto- and/or nanosecond pulsed lasers, can be combined with a dye in accordance with embodiments of the present invention to efficiently destroy the vitreous opacity, e.g. by laser-induced vapor nanobubble generation. While laser irradiation may be advantageous, irradiation by another (intense) light source is not necessarily excluded to achieve the same or similar effects.

In embodiments of the uses or methods as taught herein,
the intensity of the pulses of the laser may be at least 10⁴ W/cm²;
the number of pulses of the laser may be at least 1 laser pulse;
the pulses of the laser may have a duration in the range of at least 10 fs; and/or
the laser irradiation reaches a fluence of at least 0.1 mJ/cm² at the vitreous opacity.

In embodiments of the uses or methods as taught herein,
the intensity of the pulses of the laser may be 10⁴ to 10¹⁵ W/cm² or 10⁷ to 10¹⁵ W/cm²;
the number of pulses of the laser may be 1 to 1000 laser pulses; and/or
the pulses of the laser may have a duration in the range of 10 fs to 10 ns.

In embodiments of the methods or uses as taught herein,
the intensity of the pulses of the laser is in the range of 10⁷ to 10¹⁵ W/cm²; such as in the range of 10¹⁰ to 10¹⁵ W/cm² or in the range of 10¹² to 10¹⁴ W/cm²;
the number of pulses of the laser is in the range of 1 to 1000 laser pulses per vitreous opacity;
such as in the range of 1 to 100 laser pulses or in the range of 1 to 10 laser pulses; and/or
the duration of the pulses of the laser is in the range of 10 fs to 10 ns; such as in the range of 10 fs to 1 ps or in the range of 1 ps to 10 ns.

The laser pulses may each have a power density or intensity in the range of 10⁷ to 10¹⁵ W/cm², e.g. in the range of 10¹² to 10¹⁵ W/cm², or alternatively expressed, a fluence in the range of 10 μJ/cm² to 100 J/cm², e.g. in the range of 10 mJ/cm² to 10 J/cm² or in the range of 1 J/cm² to 10 J/cm².

The laser pulses may consist of 1 to 1000 laser pulses, such as 1 to 500 laser pulses, 1 to 100 laser pulses, 1 to 20 laser pulses, or 1 to 10 laser pulses, per vitreous opacity. The number of laser pulses may be depending on the dye and on the size, composition and shape of the vitreous opacity.

The laser pulses may have a duration in the range of 10 fs to 100 ns, for instance in the range of 10 fs to 10 ns, e.g. in the range of 10 fs to 1 ps or in the range of 1 ps to 10 ns.

The dye as taught herein advantageously allows efficient and targeted destruction of vitreous opacities.

The term “photodestruction” as used herein refers to the process of using electromagnetic radiation, such as (visible) light or near infrared radiation, to fragment tissue (e.g. the vitreous opacity). The electromagnetic radiation may be generated by a laser such as a pulsed laser.

In embodiments, the photodestruction may be laser-assisted photodestruction.

The use of the dye as taught herein may lead to fragmentation or even destruction of the vitreous opacity. Hence, further aspects or embodiments relate to the dye as taught herein for use in a method of photodestruction of a vitreous opacity.

Accordingly, further aspects or embodiments relate to a dye as taught herein for use in a method of photodestruction of a vitreous opacity in an eye of a subject.

A further aspect provides a method of photodestruction of a vitreous opacity in an eye of a subject in need of such a treatment, comprising administering a therapeutically effective amount of a dye to the subject.

Related aspects provide:
a dye for use in a method of photodestruction of a vitreous opacity in an eye of a subject.
the use of a dye for the manufacture of a medicament for photodestruction of a vitreous opacity in an eye of a subject.
the use of a dye for photodestruction of a vitreous opacity in an eye of a subject.

In certain embodiments of the methods or uses as taught herein, the method may comprise:
administering a dye to the vitreous body of the eye of the subject; and
irradiating at least part of the vitreous opacity, thereby inducing destruction of the vitreous opacity of the subject.

Accordingly, an aspect relates to a method of photodestruction of a vitreous opacity in an eye of a subject, the method comprising:
administering a dye to the vitreous body of the eye of the subject; and
irradiating at least part of the vitreous opacity, thereby inducing destruction of the vitreous opacity of the subject.

In embodiments, the treatment of a vitreous-opacity related disease may comprise performing laser irradiation of the dye as taught herein, in particular pulsed laser irradiation of the dye as taught herein. Accordingly, in embodiments, the treatment as taught herein comprises laser-assisted treatment. The terms “laser-assisted treatment”, “laser ablation treatment” or “photoablation treatment” may be used interchangeably herein.

Further aspects relate to:
a dye for use in a method of laser-assisted treatment of a vitreous opacity-related disease in a subject.
a method of laser-assisted treatment of a vitreous opacity-related disease in a subject in need of such a treatment, comprising administering a therapeutically effective amount of a dye to the subject.
the use of a dye for the manufacture of a medicament for laser-assisted treatment of a vitreous opacity-related disease in a subject.
the use of a dye for laser-assisted treatment of a vitreous opacity-related disease in a subject.

The dye as taught herein allows treatment, such as laser-assisted treatment, of a vitreous opacity-related disease.

As used herein, a phrase such as “a subject in need of treatment” includes subjects that would benefit from treatment of a given condition, particularly a vitreous opacity-related disease. Such subjects may include, without limitation, those that have been diagnosed with said condition, those prone to develop said condition and/or those in who said condition is to be prevented.

The terms “treat” or “treatment” encompass both the therapeutic treatment of an already developed disease or condition, such as the therapy of an already developed vitreous opacity-related disease, as well as prophylactic or preventive measures, wherein the aim is to prevent or lessen the chances of incidence of an undesired affliction, such as to prevent occurrence, development and progression of a vitreous opacity-related disease. Beneficial or desired clinical results may include, without limitation, alleviation of one or more symptoms or one or more biological markers, diminishment of extent of disease, stabilized (i.e., not worsening) state of disease, delay or slowing of disease progression, amelioration of the disease state, and the like. The term may encompass ex vivo or in vivo treatments.

The uses and methods as taught herein allow to administer a therapeutically effective amount of a dye as taught herein in subjects having a vitreous opacity-related disease which will benefit from such treatment. The term “therapeutically effective amount” as used herein, refers to an amount of active compound or pharmaceutical agent that elicits the biological or medicinal response in a subject that is being sought by a surgeon, researcher, veterinarian, medical doctor or other clinician, which may include inter alia alleviation of the symptoms of the disease or condition being treated.

The term “therapeutically effective dose” refers to an amount of an agent as taught herein, such as a dye, that when administered brings about a positive therapeutic response with respect to treatment of a patient having the disease or condition being treated, such as a vitreous opacity-related disease.

Appropriate therapeutically effective doses of an agent as taught herein, such as a dye, may be determined by a qualified physician with due regard to the nature of the agent, the disease condition and severity, and the age, size and condition of the patient.

In certain embodiments, the agent as taught herein, such as a dye, may be formulated into and administered as pharmaceutical formulations or compositions. Such pharmaceutical formulations or compositions may be comprised in a kit of parts.

In embodiments, the dye such as the vital dye may be comprised in a pharmaceutical formulation. The dye or pharmaceutically acceptable salts thereof can be formulated as an aqueous solution. Accordingly, an aspect relates to a pharmaceutical formulation comprising a dye as taught herein. A further aspect relates to a pharmaceutical formulation taught herein for use in a method of treating a vitreous opacity-related disease in a subject. Preferably, the subject is a human subject. The terms “pharmaceutical composition”, “pharmaceutical formulation” or “pharmaceutical preparation” may be used interchangeably herein and refer to a mixture comprising an active ingredient. The terms “composition” or “formulation” may likewise be used interchangeably herein. The terms “active ingredient” or “active component” can be used interchangeably and broadly refer to a compound or substance which, when provided in an effective amount, achieves a desired outcome. The desired outcome may be therapeutic and/or prophylactic. Typically, an active ingredient may achieve such outcome(s) through interacting with and/or modulating living cells or organisms.

The term “active” in the recitations “active ingredient” or “active component” refers to “pharmacologically active” and/or “physically active”.

The present pharmaceutical formulations may comprise in addition to the dye one or more pharmaceutically acceptable excipients.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

Pharmaceutical compositions as intended herein may be formulated for essentially any route of administration, such as without limitation, oral administration (such as, e.g., oral ingestion), parenteral administration (such as, e.g., subcutaneous, intravenous or intramuscular injection or infusion), and the like.

For example, for oral administration, pharmaceutical compositions may be formulated in the form of pills, tablets, lacquered tablets, coated (e.g., sugar-coated) tablets, granules, hard and soft gelatin capsules, aqueous, alcoholic or oily solutions, syrups, emulsions or suspensions. In an example, without limitation, preparation of oral dosage forms may be is suitably accomplished by uniformly and intimately blending together a suitable amount of the active compound in the form of a powder, optionally also including finely divided one or more solid carrier, and formulating the blend in a pill, tablet or a capsule. Exemplary but non-limiting solid carriers include calcium phosphate, magnesium stearate, talc, sugars (such as, e.g., glucose, mannose, lactose or sucrose), sugar alcohols (such as, e.g., mannitol), dextrin, starch, gelatin, cellulose, polyvinylpyrrolidine, low melting waxes and ion exchange resins. Compressed tablets containing the pharmaceutical composition can be prepared by uniformly and intimately mixing the active ingredient with a solid carrier such as described above to provide a mixture having the necessary compression properties, and then compacting the mixture in a suitable machine to the shape and size desired. Moulded tablets maybe made by moulding in a suitable machine, a mixture of powdered compound moistened with an inert liquid diluent. Suitable carriers for soft gelatin capsules and suppositories are, for example, fats, waxes, semisolid and liquid polyols, natural or hardened oils, etc.

Preferably the pharmaceutical formulation may be formulated for parenteral administration such as administration into the vitreous, e.g. by injection. In embodiments, the pharmaceutical composition may be formulated as an aqueous solution. For example, for parenteral administration, pharmaceutical compositions may be advantageously formulated as solutions, suspensions or emulsions with suitable solvents, diluents, solubilisers or emulsifiers, etc. Suitable solvents are, without limitation, water, physiological saline solution or alcohols, e.g. ethanol, propanol, glycerol, in addition also sugar solutions such as glucose, invert sugar, sucrose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. The dye or pharmaceutically acceptable salts thereof can also be lyophilized. The obtained lyophilizates can be used, for example, for injection or infusion preparation or for the production of injection or infusion preparations.

A further aspect relates to a kit of parts as taught herein for use in a method of treating a vitreous opacity-related disease in a subject. Preferably, the subject is a human subject.

The terms “kit of parts” and “kit” as used throughout this specification refer to a product containing components necessary for carrying out the specified uses or methods, packed so as to allow their transport and storage. Materials suitable for packing the components comprised in a kit include crystal, plastic (e.g., polyethylene, polypropylene, polycarbonate), bottles, flasks, vials, ampules, paper, envelopes, or other types of containers, carriers or supports. Where a kit comprises a plurality of components, at least a subset of the components (e.g., two or more of the plurality of components) or all of the components may be physically separated, e.g., comprised in or on separate containers, carriers or supports. The components comprised in a kit may be sufficient or may not be sufficient for carrying out the specified uses or methods, such that external reagents or substances may not be necessary or may be necessary for performing the methods, respectively. Typically, kits are employed in conjunction with standard laboratory equipment, such as liquid handling equipment, environment (e.g., temperature) controlling equipment, analytical instruments, etc. In addition to the dye as taught herein, optionally provided on arrays or microarrays, the present kits may also include excipients such as solvents useful in the specified uses or methods. Typically, the kits may also include instructions for use thereof, such as on a printed insert or on a computer readable medium. The terms may be used interchangeably with the term “article of manufacture”, which broadly encompasses any man-made tangible structural product, when used in the present context.

The present application also provides aspects and embodiments as set forth in the following Statements:
Statement 1. A dye for use in a method of treating a vitreous opacity-related disease in a subject.
Statement 2. A dye for use according to statement 1, wherein the method comprises:
   administering the dye to the vitreous body of an affected eye of the subject; and
   irradiating at least part of the vitreous opacity, thereby inducing destruction of the vitreous opacity in the subject.
Statement 3. The dye for use according to statement 1 or 2, wherein the dye is a vital dye; preferably wherein the dye is a vital dye approved for ophthalmological use.
Statement 4. The dye for use according to any one of statements 1 to 3, wherein the dye is a vital dye selected from the group consisting of: Indocyanine Green, Trypan Blue, Brilliant Blue, Janus green B, Gentian violet, Bromophenol Blue, Patent blue, Light Green, Fast Green, Infracyanine Green, Methylene blue, Toluidine blue, Fluorescein Sodium, Rose Bengal, and Rhodamine 6G; preferably wherein the dye is Indocyanine Green or Trypan Blue; more preferably wherein the dye is Indocyanine Green.
Statement 5. The dye for use according to any one of statements 1 to 4, wherein the dye is administered at a concentration of about 0.001 mg/ml to about 0.5 mg/ml.
Statement 6. The dye for use according to any one of statements 1 to 5, wherein the dye is a free dye or wherein the dye is conjugated to an agent and/or is comprised in a particle, such as a nanoparticle or a microparticle; preferably wherein the dye is grafted on a particle and/or wherein the dye is encapsulated in a particle.
Statement 7. The dye for use according to any one of statements 1 to 6, wherein the dye is administered to the vitreous body by intravitreal administration; preferably wherein the dye is administered to the vitreous body by intravitreal injection.
Statement 8. The dye for use according to any one of statements 1 to 7, wherein the dye is capable of diffusing in the vitreous body after administration.
Statement 9. The dye for use according to any one of statements 1 to 8, wherein the dye is capable of binding to the vitreous opacity after administration.
Statement 10. The dye for use according to any one of statements 1 to 9, wherein the dye is capable of forming vapor nanobubbles at the vitreous opacity when irradiated.
Statement 11. The dye for use according to any one of statements 1 to 10, wherein the at least part of the vitreous opacity is irradiated with electromagnetic radiation; preferably wherein the at least part of the vitreous opacity is irradiated with laser radiation; more preferably wherein the at least part of the vitreous opacity is irradiated with pulsed-laser radiation.
Statement 12. The dye for use according to statement 11, wherein:
   the intensity of the pulses of the laser may be at least 10⁴ W/cm²;
   the number of pulses of the laser may be at least 1 laser pulse;
   the pulses of the laser may have a duration in the range of at least 10 fs; and/or
   the laser irradiation reaches a fluence of at least 0.1 mJ/cm² at the vitreous opacity.
Statement 13. A method of photodestruction of a vitreous opacity in an eye of a subject, the method comprising:
   administering a dye to the vitreous body of the eye of the subject; and
   irradiating at least part of the vitreous opacity, thereby inducing destruction of the vitreous opacity of the subject.
Statement 14. The method according to statement 13, wherein:
   the dye is a vital dye;
   the dye is a vital dye approved for ophthalmological use;
   the dye is a vital dye selected from the group consisting of: Indocyanine Green, Trypan Blue, Janus green B, Gentian violet, Bromophenol Blue, Patent blue, Brilliant Blue, Light Green, Fast Green, Infracyanine Green, Methylene blue, Toluidine blue, Fluorescein Sodium, Rose Bengal, and Rhodamine 6G; preferably the dye is Indocyanine Green or Trypan Blue; more preferably the dye is Indocyanine Green;
   the dye is administered at a concentration of about 0.001 mg/ml to about 0.5 mg/ml;
   the dye is a free dye;
   the dye is conjugated to an agent;
   the dye is comprised in a particle, such as a nanoparticle or a microparticle; preferably wherein the dye is grafted on a particle and/or wherein the dye is encapsulated in a particle;
   the dye is administered to the vitreous body by intravitreal administration; preferably the dye is administered to the vitreous body by intravitreal injection;
   the dye is capable of diffusing in the vitreous body after administration;
   the dye is capable of binding to the vitreous opacity after administration;
   the dye is capable of forming vapor nanobubbles at the vitreous opacity when irradiated;
   the at least part of the vitreous opacity is irradiated with electromagnetic radiation;
   preferably wherein the at least part of the vitreous opacity is irradiated with laser radiation; more preferably wherein the at least part of the vitreous opacity is irradiated with pulsed-laser radiation;
   the intensity of the pulses of the laser may be at least 10⁴ W/cm²;
   the number of pulses of the laser may be at least 1 laser pulse;
   the pulses of the laser may have a duration in the range of at least 10 fs; and/or
   the laser irradiation reaches a fluence of at least 0.1 mJ/cm² at the vitreous opacity.
Statement 15. Use of a dye for photodestruction of a vitreous opacity in an eye of a subject.
Statement 16. A vital dye for use in a method of treating a vitreous opacity-related disease in a subject.
Statement 17. A vital dye for use according to statement 16, wherein the method comprises:
   administering the vital dye to the vitreous body of an affected eye of the subject; and
   irradiating at least part of the vitreous opacity, thereby inducing destruction of the vitreous opacity in the subject.
Statement 18. The vital dye for use according to statement 16 or 17, wherein the vital dye is a vital dye approved for ophthalmological use.
Statement 19. The vital dye for use according to any one of statements 16 to 18, wherein the vital dye is selected from the group consisting of: Indocyanine Green, Trypan Blue, Brilliant Blue, Janus green B, Gentian violet, Bromophenol Blue, Patent blue, Light Green, Fast Green, Infracyanine Green, Methylene blue, Toluidine blue, Fluorescein Sodium, Rose Bengal, and Rhodamine 6G; preferably wherein the vital dye is Indocyanine Green or Trypan Blue; more preferably wherein the vital dye is Indocyanine Green.
Statement 20. The vital dye for use according to any one of statements 16 to 19, wherein the vital dye is administered at a concentration of about 0.001 mg/ml to about 0.5 mg/ml.
Statement 21. The vital dye for use according to any one of statements 16 to 20, wherein the vital dye is a free dye or wherein the vital dye is conjugated to an agent and/or is comprised in a particle, such as a nanoparticle or a microparticle; preferably wherein the vital dye is grafted on a particle and/or wherein the vital dye is encapsulated in a particle.
Statement 22. The vital dye for use according to any one of statements 16 to 21, wherein the vital dye is administered to the vitreous body by intravitreal administration; preferably wherein the vital dye is administered to the vitreous body by intravitreal injection.
Statement 23. The vital dye for use according to any one of statements 16 to 22, wherein the vital dye is capable of diffusing in the vitreous body after administration.
Statement 24. The vital dye for use according to any one of statements 16 to 23, wherein the vital dye is capable of binding to the vitreous opacity after administration.
Statement 25. The vital dye for use according to any one of statements 16 to 24, wherein the vital dye is capable of forming vapor nanobubbles at the vitreous opacity when irradiated.
Statement 26. The vital dye for use according to any one of statements 16 to 25, wherein the at least part of the vitreous opacity is irradiated with electromagnetic radiation; preferably wherein the at least part of the vitreous opacity is irradiated with laser radiation; more preferably wherein the at least part of the vitreous opacity is irradiated with pulsed-laser radiation.
Statement 27. The vital dye for use according to statement 26, wherein:
   the intensity of the pulses of the laser may be at least 10⁴ W/cm²;
   the number of pulses of the laser may be at least 1 laser pulse;
   the pulses of the laser may have a duration in the range of at least 10 fs; and/or
   the laser irradiation reaches a fluence of at least 0.1 mJ/cm² at the vitreous opacity.
Statement 28. A method of photodestruction of a vitreous opacity in an eye of a subject, the method comprising:
   administering a vital dye to the vitreous body of the eye of the subject; and
   irradiating at least part of the vitreous opacity, thereby inducing destruction of the vitreous opacity of the subject.
Statement 29. The method according to statement 28, wherein:
   the vital dye is a vital dye approved for ophthalmological use;
   the vital dye is selected from the group consisting of: Indocyanine Green, Trypan Blue, Janus green B, Gentian violet, Bromophenol Blue, Patent blue, Brilliant Blue, Light Green, Fast Green, Infracyanine Green, Methylene blue, Toluidine blue, Fluorescein Sodium, Rose Bengal, and Rhodamine 6G; preferably the vital dye is Indocyanine Green or Trypan Blue; more preferably the vital dye is Indocyanine Green;
   the vital dye is administered at a concentration of about 0.001 mg/ml to about 0.5 mg/ml;
   the vital dye is a free dye;
   the vital dye is conjugated to an agent;
   the vital dye is comprised in a particle, such as a nanoparticle or a microparticle; preferably wherein the vital dye is grafted on a particle and/or wherein the vital dye is encapsulated in a particle;
   the vital dye is administered to the vitreous body by intravitreal administration; preferably the vital dye is administered to the vitreous body by intravitreal injection;
   the vital dye is capable of diffusing in the vitreous body after administration;
   the vital dye is capable of binding to the vitreous opacity after administration;
   the vital dye is capable of forming vapor nanobubbles at the vitreous opacity when irradiated;
   the at least part of the vitreous opacity is irradiated with electromagnetic radiation; preferably wherein the at least part of the vitreous opacity is irradiated with laser radiation; more preferably wherein the at least part of the vitreous opacity is irradiated with pulsed-laser radiation;
   the intensity of the pulses of the laser may be at least 10⁴ W/cm²;
   the number of pulses of the laser may be at least 1 laser pulse;
   the pulses of the laser may have a duration in the range of at least 10 fs; and/or
   the laser irradiation reaches a fluence of at least 0.1 mJ/cm² at the vitreous opacity.
Statement 30. Use of a vital dye for photodestruction of a vitreous opacity in an eye of a subject.

The above aspects and embodiments are further supported by the following non-limiting examples.

### EXAMPLES

### Example 1: Investigation of Indocyanine Green, Trypan Blue, and Nanoparticles Comprising Indocyanine Green According to Embodiments of the Invention to Destroy Vitreous Opacities

This example investigated the capacity of two FDA-approved photosensitizers, Indocyanine Green (ICG) and Trypan Blue (TB), to destroy vitreous opacities. First, the efficacy of free ICG on the destruction of artificial floaters of type I collagen fibers was compared with various kinds of ICG-loaded nanoparticles. Secondly, the efficacy of free ICG on vitreous opacities obtained after vitrectomy in patients was compared with various kinds of ICG-loaded nanoparticles. Nanoparticles of ICG are interesting to limit the penetration of ICG in the retina by prolonging the residence time in the vitreous and therefore limit acute toxicity at the level of the retina. Indeed, the inner limiting membrane, which covers the retina, has pores which do not allow the crossing of nanoparticles with a size superior to 100 nm. In this study, different types of ICG-loaded nanomedicines, i.e. polymer nanoparticles, albumin nanoparticles, and liposomes with various charges of surface were prepared and their effect was compared with free ICG and free TB in terms of floater destruction.

Material and Methods

Chemicals

The following chemicals were used as obtained: poly(allylamine) (PAH; 17 000 g/mol) (Sigma-Aldrich, St. Louis, USA); Human serum albumin (HSA) (Sigma-Aldrich, St. Louis, USA); Indocyanine Green (ICG) (Sigma-Aldrich, St. Louis, USA); Trypan Blue (TB) (Sigma-Aldrich, St. Louis, USA); rat tail collagen type I acid solution (Sigma-Aldrich, St. Louis, USA); ethanol (Chem-Lab NV, Zedelgem, Belgium) and DMSO (Sigma-Aldrich, St. Louis, USA).

Cell Culture

MIO-M1 cells were cultured in DMEM Glutamax medium (Thermo Fisher Scientific, Waltham, USA). The medium contained 10% foetal bovine serum (FBS) (Thermo Fisher Scientific, Waltham, USA), 100 IU/ml penicillin (Gibco®-Invitrogen life technologies, Walthan, USA), 100 μg/ml streptomycin (Gibco®-Invitrogen life technologies, Walthan, USA) and 2 mM L-Glutamin (Gibco®-Invitrogen life technologies, Walthan, USA). Before use, the medium was passed through a 0.2 μm PES membrane vacuum filter (VWR, Radnor, USA). The cells were plated in polystyrene cell culture flasks (surface=75 cm²) (VWR, Radnor, USA) and placed in the incubator (37° C., 5% CO₂) (Thermo Fisher Scientific, Waltham, USA) until confluence was reached (i.e. the cells forming a monolayer covering 80%-90% of the culture flask). The morphology was checked by microscopy (VWR, Radnor, USA). After reaching the confluence (more or less after 5 days) and checking the morphology, cells were split in new culture flasks. The medium of the confluent flask was removed and then the cells were washed with preheated PBS (Thermo Fisher Scientific, Waltham, USA). The Müller cells (adherent cells) were treated with 3 ml trypsin (0.25%) (Gibco®-Invitrogen life technologies, Walthan, USA) and incubated for 5 minutes to detach the cells. The detachment of the cells was checked with microscopy. 7 ml culture medium was added to the detached cells and the total content (±10 ml) was transferred to a falcon tube (15 ml) (Nerbe plus GmbH, Winsen, Germany). To remove the (toxic) trypsin, the falcon tube was centrifuged (5 minutes, 0.2 rcf) and a clear pellet was visible. The supernatant was removed and the pellet was redispersed in culture medium (around 4 ml). 9 ml of cell culture medium was placed in a culture flask and 1 ml of the cell suspension was added. The flasks were homogenized and placed in the incubator. All the liquids (PBS, culture medium, trypsin) were preheated at 37° C. in a pellet bath.

Preparation of ICG-Nanoparticles

PAH-ICG Nanoparticles

A poly(allylamine) hydrochloride (PAH) solution with a concentration of 2 mg/ml and a Na₂HPO₄ (disodium phosphate) (Merck, Leuven, Belgium) with a molar concentration of 0.005 M were prepared. 200 μL of the PAH solution was mixed with 1200 μL of the Na₂HPO₄ solution. Subsequently 12 ml deionized water was added and vortexed for 10 seconds. Lastly 1200 μL of a 1 mg/ml aqueous ICG solution was added to the solution and vortexed for 10 seconds. All the solutions were precooled at 4° C. The suspension was aged for 2 hours at 4° C. The total amount of the suspension was divided in different Eppendorf tubes (14 Eppendorf tubes with 1 ml) and placed in the centrifuge (1 hour, 1000 G rcf) (Beckman Coulter, California, USA). After centrifugation, the supernatant was removed and kept aside. The pellet in each Eppendorf was washed and redispersed with the same volume of PBS solution. Eventually the solution was centrifuged again (same conditions), the supernatant was also removed and kept aside. All the pellets divided over the different Eppendorf tubes, were brought together with 200 μL PBS solution. The nanoparticles were stored at 4° C. protected from light. The same nanoparticles were prepared without ICG. The amount of ICG solution was replaced by deionized water.

HSA-ICG Nanoparticles

ICG and HSA were dissolved in a 50 mM GSH (Sigma-Aldrich, St. Louis, USA) solution at a concentration of 20 mg/ml and 80 mg/ml respectively. 1 ml ICG solution was mixed with 1 ml of the HSA solution. Subsequently, 2 ml ethanol was added to precipitate the HSA-ICG NPs. The suspension was magnetically stirred (Heidolph Instruments GmbH & CO. KG, Schwabach, Germany) at room temperature for 30 minutes. Then, the suspension was transferred with a syringe into a dialyse cassette of 8 ml with a cut-off of 10,000 Da (Thermo Fisher Scientific, Waltham, USA). After the suspension was transferred, the remaining air was removed from the cassette with another syringe. The dialyse cassette was placed with a Slide-A-Lyzer (to prevent the cassette from sinking) (Thermo Fisher Scientific, Waltham, USA) in a large beaker (around 1 L) filled with deionized water and the beaker was placed on a magnetic stirrer (to create a flow in the beaker) for 24 hours at 4° C. After the dialyse, the suspension was removed out of the cassette with a syringe and stored in a falcon tube protected from light at 4° C.

Liposomes Encapsulating ICG (Lip-ICG)

Positively charged liposomes loaded with ICG were prepared by mixing distearoylphosphatidylcholine (DSPC) and 1, 2-Dioleoyl-3-Trimethylammonium-Propane (DOTAP) (1:1 molar ratio) using the thin film rehydration method, as described by Lajunen et al., 2016, Mol. Pharm., 13, 2095-2107. Obtained liposomes were then sonicated for 1 min using a tip sonicator and purified by dialysis.

Characterization of Nanoparticles

Encapsulation Efficiency

The ICG loading efficiency was determined based on a calibration curve of free ICG obtained by UV-vis spectrometry (NanoDrop 2000C, Thermo Fisher Scientific, Waltham, USA) for both nanoparticles.

For the PAH-ICG NPs, several ICG concentrations (0.5 μg/ml, 1 μg/ml, 2.5 μg/ml, 5 μg/ml and 7.5 μg/ml) were prepared in triplicate from a stock solution of free ICG and diluted in PBS (each concentration was prepared in triplicate). PBS was used to measure the blank and the absorbance was determined at 780 nm. The supernatant that was removed, was kept aside for determining the loss of free ICG. The supernatant of the first washing step was diluted 1/10 in PBS, the second supernatant did not require any dilution. The absorbance of both supernatants was measured at 780 nm. Based on the absorbance, the concentration of free ICG in the supernatants was determined and the encapsulation efficiency for the PAH-ICG NPs (EE₁) was calculated using following equation:

EE₁%=(mass ICG start−total mass ICG lost)/(mass ICG start)×100

Some modifications were made to establish the calibration curve and to determine the encapsulation efficiency for the HSA-ICG NPs. The measurements (blank, calibration curve and the HSA-ICG NPs) were performed in DMSO/H20 (9:1, V/V). Free ICG was diluted in a concentration range from 0.25 ug/ml to 5 ug/ml (0.25 ug/ml, 0.50 ug/ml, 1.0 ug/ml, 2.5 ug/ml and 5.0 ug/ml), and each concentration was prepared three times. The absorbance was measured at 780 nm and the calibration curve was compiled. The nanoparticles were diluted 1/500 in DMSO/H2O (9:1, V/V) and subsequently the absorbance was measured at 780 nm. DMSO was used to break the particles, which releases the ICG. The amount of free ICG was then measured by UV-vis spectrometry. The encapsulation efficiency for the HSA-ICG NPs (EE₂) was calculated by following equation:

EE₂%=(mass ICG end)/(mass ICG start)×100

### Size and Zeta-Potential

The PAH-ICG NPs and the HSA-ICG NPs were diluted 1/100 in deionised water and measured by DLS and electrophoretic mobility respectively at 25° C. using a Nanosizer (Malvern Instruments, Malvern, UK) to determine the size and zeta-potential. For the PAH-ICG NPs, these measurements were performed before the particles were stored for 2 hours at 4° C. and after the centrifugation steps where the free ICG was removed. The size and zeta-potential of the HSA-ICG NPs were measured after the dialysis. The dispersions were prepared in a laminar air flow cabinet to avoid dust into the samples. 1 ml of the dispersion was transferred into a folded capillary cell.

Toxicity Studies on Human Immortalized Müller Cells (MIO-M1)

Free ICG

To test the toxicity of free and nano-encapsulated ICG, we wanted to seed 7,000 Müller cells in each well of a 96-well plate (VWR, Radnor, USA). The cells were counted with a Bürker counting chamber (BRAND, Wertheim, Germany) to obtain the proper number of cells. The cells were treated the same as in section 3.2. After redispersing the cell pellet (obtained after centrifugation) in 4 ml culture medium, 50 μl was transferred in an Eppendorf tube to count the cells. 100 μL trypan blue (an azo dye to stain the death cells) (Sigma-Aldrich, St. Louis, USA) was added to the Eppendorf and 10 μL of this mixture was transferred on both sides of the Bürker counting chamber. The non-coloured cells were counted in 6 big squares (3 on each side of the chamber); each big square contained 0.1 μL mixture. The dilution factor by trypan blue (1/3) and a factor to go from 0.1 μL to 1 μL (factor 10,000) were taken into account to calculate the number of cells per ml. Based on the amount of cells, a dilution was prepared to obtain 7,000 cells in 200 μL cell suspension for each well. Each well was filled with 200 μL of the diluted cell suspension and placed in the incubator for 24 hours.

After 24 hours incubation time, the cell medium was removed and treated with different concentrations of free ICG. Out of a stock solution of free ICG, dilution series were prepared in cell medium: 0.1 mg/ml, 0.3 mg/ml, 0.5 mg/ml, 0.75 mg/ml and 1 mg/ml. The control contained cell medium and an amount of deionized water, namely the same amount of water as the amount of ICG solution used to obtain a 1 mg/ml concentration in cell medium was added (most stringent concentration). The cell medium was removed out of the 96-well plate and replaced by 200 μL of each solution (control and five different concentrations of ICG). Each condition was performed in quintuple. The well plate was covered in aluminium foil and incubated for 24 hours at 37° C.

To perform the MTT assay, a stock solution of 5 mg/ml MTT reagent (Sigma-Aldrich, St. Louis, USA) was prepared in PBS. For one well, 30 μL MTT reagent (5 mg/ml) was mixed with 200 μL cell medium. The solution was carefully removed in each well, then each well was washed twice with 100 μL PBS and 200 μL of the MTT solution was added. The control wells were treated the same. The cells were incubated for 3 hours at 37° C., wrapped in aluminium foil. After incubation, the solution in the wells was removed again and replaced by 100 μL DMSO. The 96-well plate was covered in aluminium foil and placed on an orbital shaker (Heidolph Instruments GmbH & CO. KG, Schwabach, Germany) for 30 minutes. The MTT assay was performed with the Victor³ plate reader (PerkinElmer, Waltham, USA). The absorbance was determined at 595 nm. The metabolic activity of the cells, treated with the ICG solution, was compared to the control. After receiving the results of the first MTT-assay, it was noticed that ICG was stuck on the 96-well plate (even after 2 washing steps with PBS) and contributed to the absorbance measured at 595 nm with the Victor³ plate reader. To solve this problem, additional controls were implemented to take the background of ICG into account. The additional wells were treated with the same dilutions of free ICG (0.1 mg/ml, 0.3 mg/ml, 0.5 mg/ml, 0.75 mg/ml and 1 mg/ml), but after 24 hours incubation, cell medium was added to the additional wells instead of the MTT reagent. In this way, it was possible to determine the absorbance of the ICG that was stuck onto the 96-well plate. The background absorbance of the ICG was subtracted from the absorbance obtained for the wells that were incubated with the MTT-reagent and then compared to the control. This experiment was performed in triplicate.

Nano-Encapsulated ICG

The optimized experiment was performed for PAH-ICG NPs, HSA-ICG NPs and LIP-ICG. A few adaptations were made. For both types of nanoparticles, each condition was performed in triplicate. Another problem for the HSA-ICG NPs arose: the obtained concentration of ICG after dialysis was relatively low in the HSA-ICG NPs, therefore the dilution series contained much less cell medium. For this reason, each concentration of HSA-ICG NPs had his own control: the amount of water added to the cell medium of the control was specific for each concentration (i.e. not only the most stringent condition).

Poly(Allylamine) Hydrochloride

To test the toxicity of the PAH polymer itself (i.e. without ICG), the amount of polymer in which the cells were exposed was calculated for each concentration of the PAH-ICG NPs. The same experiment was performed as described above, except the additional control that determined the background signal of ICG was left out (because no ICG was used).

Photo-Ablation of Artificial and Human Vitreous Opacities

Preparation of Artificial Floaters (Type I Collagen Fibers)

Collagen fibers were prepared from type I collagen from the rat tail (GIBCO; concentration of 3 mg/ml) as described in Sauvage et al., 2019, ACS Nano, 13, 8401-8416. 5 ml of PBS was pipetted in a 15 ml falcon tube. 330 μL of the PBS solution was replaced by 330 μL collagen type I and the solution was vortexed. Sodium hydroxide (0.1 M) (VWR, Radnor, USA) was added to the acid solution of collagen to adjust pH to 7.4. The solution was vortexed again and placed for one hour in the incubator at 37° C. The final concentration of the collagen was 0.2 mg/ml.

Human Vitreous Opacities

Samples of the vitreous containing human floaters were obtained from VMR institute (Huntington Beach, Calif., USA) where vitrectomies of the patients were performed. The human opacities were diluted one on one (v/v) with a stock solution of free ICG with a concentration of 1 mg/ml, so that the final ICG concentration was 0.5 mg/ml. The samples were placed on a glass bottom dish and covered with a cover glass.

Laser Treatment of Artificial and Human Opacities

First, dark-field microscopy imaging was performed to locate and align the nanosecond laser (Opolette HE 355 LD, OPOTEK Inc., CA, USA) on the artificial collagen fibers in the sample. Samples of either artificial or human opacities treated with TB, ICG and ICG nanoparticles were then illuminated with the laser (<7 ns). The wavelength of the laser light was 561 nm and the energy was set at ±800 μJ. A beam expander (#GBE05-A, Thorlabs) combined with iris diaphragm (#D37SZ, Thorlabs) were used to adjust the diameter of the laser beam to 150 μm (so that 800 μJ corresponded to a laser fluence of 4.5 J/cm²). The laser pulse energy was monitored by an energy meter (J-25 MB-HE&LE, Energy Max-USB/RS sensors, Coherent) synchronized with the pulsed-laser. The set-up was made in such way to illuminate the sample shot by shot. Videos of the sample were made during illumination with NIS software. Same experiments were performed with human opacities Comparison Between Free ICG, PAH-ICG NPs and HSA-ICG NPs in Water and Bovine Vitreous.

Quantification of the Number of Bubbles as a Function of Laser Fluence

The same laser set-up as described in the previous section was used to generate VNBs. VNBs scatter light in an efficient way, therefore they can be easily detected by dark-field microscopy. Videos were made of the generation of VNBs with a single laser pulse at two different laser energies (100 μJ and 800 μJ). This experiment was performed in water and bovine vitreous. Number of bubbles was then plotted as a function of laser fluence using graphpad prism.

In Vivo Imaging and Photo-Ablation of Collagen Fibers

Animal Preparation

All the animal experiments were performed under the guidelines of the Association for Research in Vision and Ophthalmology (ARVO) Statement on the Use of Laboratory Animals in Ophthalmic and Vision Research. The experimental protocols were reviewed and approved by the Institutional Animal Care and Use Committee (IACUC) of the University of Michigan (Protocol PRO00008566, PI Paulus).

A total of 24 New Zealand White rabbits (age 3-6 months; weight 2.45-3.15 kg; both genders) were used. The rabbits were randomly divided into 7 groups: a control group A treated with laser only (n=3), group B received an intravitreal (IVIT) injection of ICG (n=9), group C received an IVIT injection of ICG labeled collagen fibers (n=3), group D was IVIT injected with collagen fibers followed by an IVIT injection of ICG (n=3), group E was IVIT injected with HA-AuNPs labeled floaters (n=3), and group F was IVIT injected with floaters followed by an IVIT injection of HA-AuNPs (n=3). During the in vivo experiments, animal conditions such as mucous membrane color, heart rate, body heat, and respiratory rate were monitored every 15 minutes. The animals were anesthetized by intramuscular injection of ketamine (40 mg/kg) and xylazine (5 mg/kg). A drop of tropicamide (1%) and phenylephrine hydrochloride (2.5%) was applied into the rabbit eyes to dilate their pupils at 15 min before the imaging procedures. For topical anaesthesia, a drop of topical tetracaine (0.5%) was used. To avoid corneal dehydration during the experiment, a lubricant solution (Systane, Alcon Inc., TX, USA) was added into the eye every minute using a syringe with plastic needle. To maintain the anaesthesia, a dose of ketamine (13 mg/kg) was injected every 45 min. The animal's body heat was maintained using a circulation heat blanket.

Intravitreal Injections

First, to visualize the retina of the rabbit eyes, a plastic contact lens was placed on the cornea. When the target location in the vitreous was selected under the microscope, the rabbits under anaesthesia were subsequently intravitreally injected. The intravitreal injections were performed using a 27 gauge needle. In one series of experiments 40 μL of ICG-fibers (which are collagen fibers treated with ICG) was injected; the concentration of collagen in the dispersions was 0.02 mg/mL. In another series of experiments, 40 μL of collagen fibers (0.02 mg/mL) were intravitreally injected. 5 days after the injection of the fibers, ICG (0.25-1.25 mg/ml) was injected in the vitreous (40 μl). The position of the fibers was monitored by OCT so that the intravitreal injection of ICG could be performed close the area where the fibers were located. Three days after the injection of ICG, laser treatment was performed.

Color Fundus Photography, Fluorescence Imaging, PAM- and OCT-Imaging

The rabbits were monitored one minute after intravitreal injection of the collagen fibers and at day 4 post intravitreal injection of ICG. Rabbits intravitreally injected with ICG only were followed up for 14 days post injection. The rabbit eyes were assessed by color fundus photography, fluorescence imaging, optical coherence tomography (OCT) and photoacoustic microscopy (PAM), as discussed below. The rabbit's head and body were positioned in two different platforms to minimize breathing and other motion artifacts. The same scanning areas for PAM- and OCT-imaging were monitored by the fundus camera which was integrated in the OCT system. To detect the photoacoustic signal, the ultrasound transducer was placed in contact with the conjunctiva, allowing it to move freely in 3D while not applying any physical pressure on the rabbit eyes. The scanning areas (i.e. areas containing the injected fibers) were selected by the fundus camera and captured by PAM.

Color fundus photography was performed using a 50-degree color fundus photography system (Topcon 50EX, Topcon Corporation, Tokyo, Japan). The retina fundus was captured using an EOS 5D camera (resolution of 5472×3648 pixels with a pixel size of 6.55 μm²; Canon, Japan). Several positions of the eye were imaged including the optic nerve, the superior retina above the optic disc, the inferior retina below the optic disc, the temporal medullary ray and the nasal medullary ray. Fluorescence imaging was done with the Topcon 50EX system using appropriate excitation and emission filters.

For photoacoustic microscopy (PAM) and optical coherence tomography (OCT) imaging a home-built integrated PAM and OCT system was developed to track the location of the fibers in the vitreous. Briefly, for PAM, a tunable nanosecond pulsed laser produced by a solid-state Q-switched Nd:YAG laser (NT-242, Ekspla, Lithuania) was used as a light source. The optical wavelength could be adjusted (405-2600 nm), the pulse repetition rate was 1 kHz and the pulse duration 3-5 ns. The output laser light was spread through the iris, filtered and collimate to form a homogeneous beam size of 2 mm. Then, the laser light was passed through a galvanometer and telescope consisting in a scan lens and an ocular lens and focused on the fundus of the retina with an estimated diameter of 20 μm. To detect the photoacoustic (PA) signal, a custom-made needle shape ultrasound transducer was used (center frequency of 27 MHz, two-way bandwidth—60%, Optosonic Inc., Arcadia, Calif., USA). The detected PA signals were amplified using a 1.4 dB preamplifier (AU-1647, L3 Narda-MITEQ, NY). Then, the analog data were converted into digital signals and digitized at a sampling rate of 500 MHz using a DAQ card (PX1500-4, Signatec Inc., Newport Beach, Calif.).

For PAM-imaging, light of 578 nm (to detect retinal and choroidal vessels) and 800 nm (to detect ICG) with an average energy of 80 nJ was shined into the eyes. This is about half of the maximal energy of a single laser pulse (˜160 nJ at 570 and 800 nm) which might be applied on the retina, as defined by the American National Standards Institute (ANSI). By using the full-width at half-maximum (FWHM) of line spread functions (LSFs) and A-line signal (one-dimensional point spread function), the lateral and axial resolution equaled 4.1 μm and 37.0 μm, respectively. Both 2D and 3D PAM-images could be obtained with an acquisition time of 65 s by using an optical scanning galvanometer.

The OCT setup used in this study was built using a commercially available spectral domain Ganymede-II-HR OCT device (Thorlabs, Newton, N.J.) to which a dispersion compensation glass and an ocular lens were added. To excite the sample, two super luminescent diodes with central wavelengths of 846 nm and 932 nm were used. The incident light beam was coaxially aligned with the PAM laser beam, allowing to obtain both PAM and OCT at the same location and co-registering the OCT and PAM-images on the same orthogonal imaging plane. The OCT lateral and axial resolutions were 3.8 am and 4.0 μm, respectively. A cross-sectional B-scan OCT image can be obtained within 0.103 seconds with a resolution of 512×1024 A-lines at the scanning rate of 36 kHz. 3D volumetric OCT images with a volume of 4.5×4.5×1.8 mm³ (512×512×1024 pixels) were obtained within 2 min (with average rate of 3 times).

Laser Treatment of Rabbit Eyes In Vivo

Before laser treatment, the rabbit's eyes were imaged by PAM and OCT (see section above). For laser treatment, anesthetized rabbits were kept on a custom-built stabilization platform. After acquiring the images and locating the opacities (collagen fibers) by OCT, areas in the eyes (4.5×4.5 mm²) were illuminated with laser pulses (<7 ns; 1.9 J/cm²) NT-242, Ekspla, Lithuania) of 800 nm (ICG) to destroy the collagen fibers; the step size of the scanning laser was 9 μm, the beam size equaled 20 μm. During the treatment, real-time OCT was active to monitor the position of the collagen fibers. The 4.5×4.5 mm² area was laser scanned several times (3-7 times) until the collagen fibers were completely destroyed. After the laser treatment, PAM and fundus images were performed to evaluate the potential damage of retinal vessels. Also, rabbit's vital signs were monitored and recorded until the animals fully recovered from anaesthesia.

In Vivo Safety Evaluation

Treated eyes were ophthalmologically evaluated immediately after the laser treatment and followed up for 1 month. Anterior segment structures such as the eyelids, iris, conjunctiva, cornea, anterior chamber and lens were comprehensively examined using slit lamp bio-microscopy (SL120, Carl Zeiss, Germany). In addition, posterior segment structures (i.e., the vitreous, optic nerve and retina) were assessed using a contact fundus lens (Volk Optical Inc, Mentor, Ohio, USA).

Statistical Analysis

The One-way ANOVA was used to calculate statistical significance. Data were considered significantly different when p<0.05.

Results

Characterization and Toxicity Screening of ICG-Loaded Nanoparticles

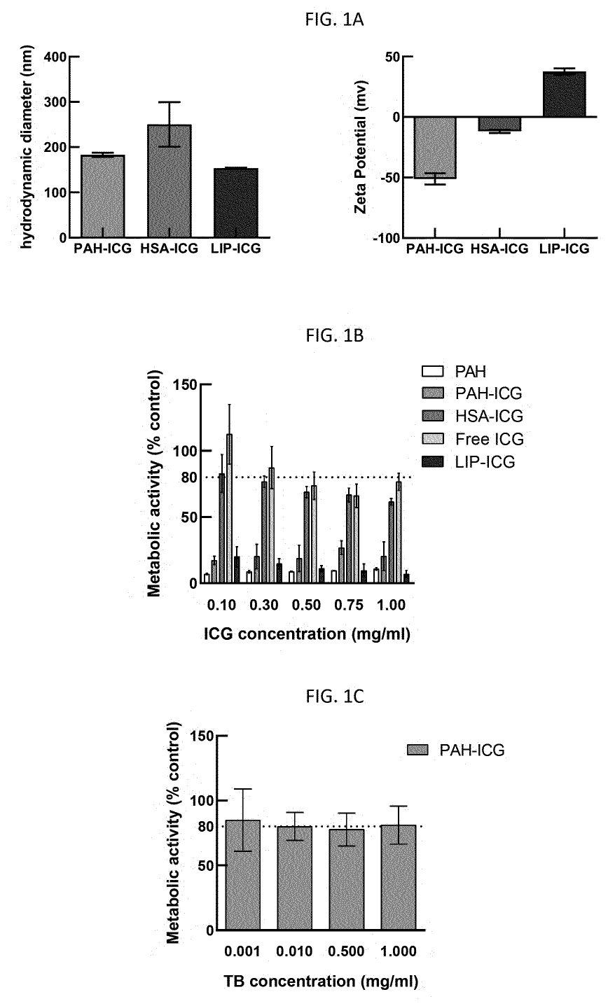

After preparation, the size of Poly(allylamine) hydrochloride (PAH)-ICG nanoparticles, Human serum albumin (HAS)-ICG nanoparticles, and Liposomes encapsulating ICG (LIP-ICG) were 183, 250 and 153 nm, respectively. The obtained sizes were, therefore, more than 100 nm (>100 nm) as required for a prolonged time in the vitreous since particles with a size>100 nm cannot penetrate the inner limiting membrane (FIG. 1A, left). The zeta potential of PAH-ICG, HSA-ICG, and LIP-ICG were found to be −51, −11, and +37 my (FIG. 1A, right).

To have a first indication of the toxicity of free and nano-encapsulated ICG, MTT assays were performed on immortalized human Müller cells. This type of cell is directly located at the vitreoretinal interface and is, therefore, a good model for toxicity screening of the retina. A first observation was PAH-ICG was found to be toxic with decreased metabolic activity (FIG. 1C). This toxicity can be attributed to PAH, as PAH nanoparticles were found to be toxic, as well. Free ICG showed rather acceptable toxicity up to a concentration of 0.5 mg/ml, and cells treated with HSA-ICG showed a slight decrease of metabolic activity compared to free ICG for all the studied concentrations (FIG. 1B).

Comparison Between Free and Nano-Encapsulated ICG for the Destruction of Type I Collagen Fibers after Illumination with the Nanosecond Laser

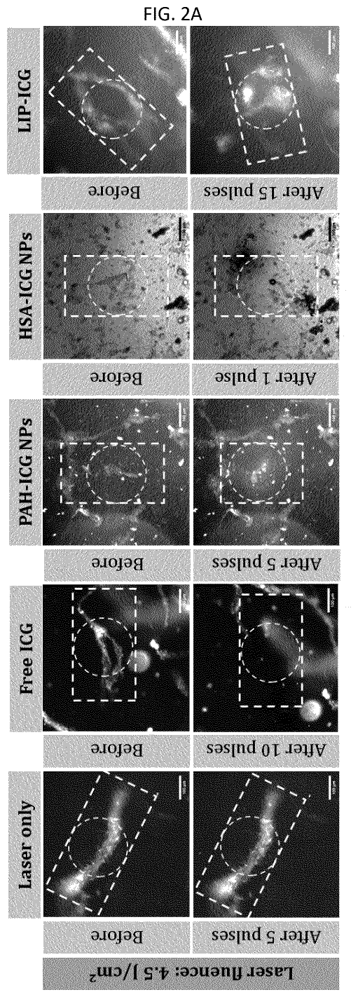

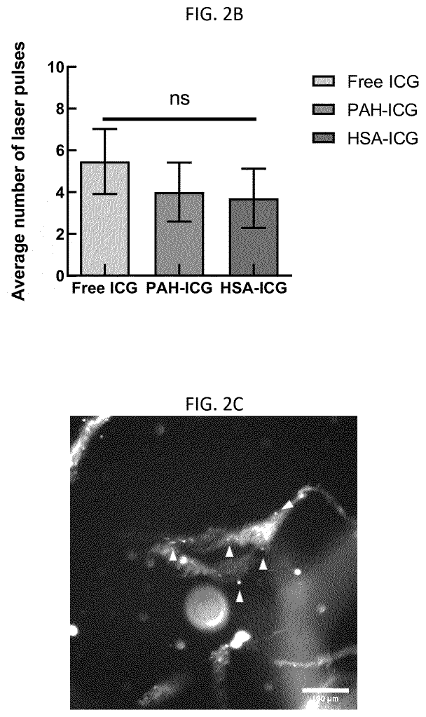

Type I collagen fibers are well-suited as an in vitro model to form artificial floaters. Therefore, the capacity of ICG-loaded NPs and free ICG was investigated to destroy type I collagen fibers in water after illumination with the nanosecond laser. From the results of the viability assays, a concentration of 0.5 mg/ml was used for which the in vitro toxicity of free ICG was acceptable (FIG. 1B). The first observation was that, in the absence of free ICG or nanoparticles, the laser was not sufficient to break type I collagen fibers (data not shown). However, when fibers were mixed with free ICG, HSA-ICG, or PAH-ICG, they could be destroyed after illumination with the nanosecond laser (4.5 J/cm²) with a number of pulses less than 10 (<10). Nevertheless, LIP-ICG were not found to be efficient in destroying the fiber though some bubbles could be observed during the pulse. After five pulses, the structure of the fiber was affected; however, after 15 pulses, no effects could be observed (FIG. 2A, last column).

To get better insights into the efficacy of nanoparticles, the average number of pulses needed to destroy one fiber was measured (FIG. 2B). It appears that for free and ICG-loaded nanoparticles, an average number of 4-5 pulses was sufficient to destroy one fiber (FIG. 2B).

It is also important to note that some aggregates could be visualized with HSA and PAH nanoparticles during these experiments, whereas it was not the case with free ICG and LIP-ICG.

Another interesting observation was that VNBs could be observed at the level of the fiber with free ICG, whereas for ICG-loaded nanoparticles, they could be observed at different places inside the laser beam.

ICG can Trigger VNB on Type I Collagen Fiber

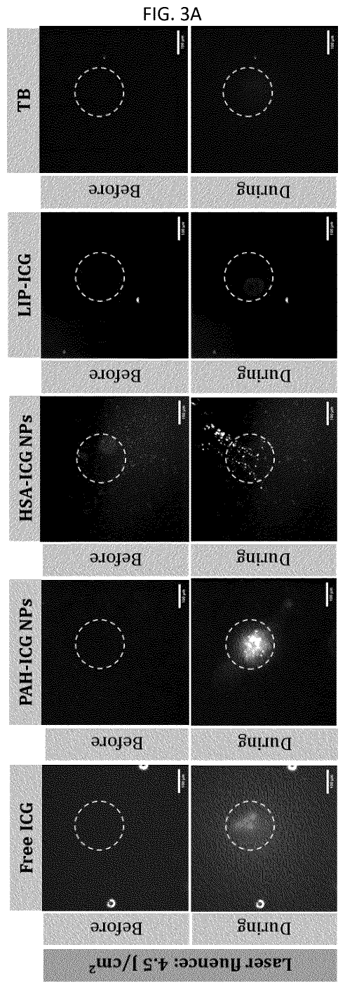

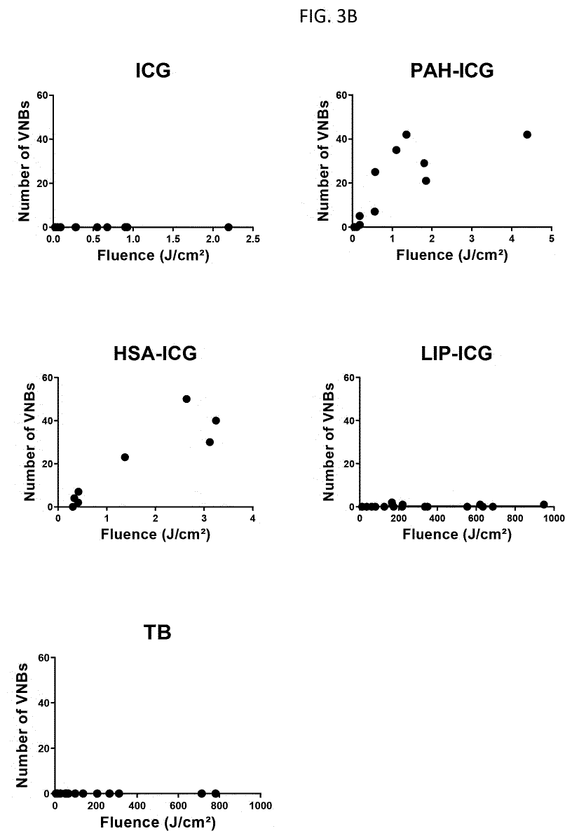

To check whether free ICG can trigger VNBs in a targeted manner (i.e. on collagen fibers), dark field microscopy images of free ICG and nano-encapsulated ICG was performed in water and bovine vitreous. The first observation is that for some particles bright spots could be observed (FIG. 3A). The number of VNBs generated in water was plotted as a function of laser intensities (FIG. 3B). It appears clearly that free ICG and free TB did not produce VNBs in water at this fluence (FIG. 3B). Hence, free ICG was interesting due to its capacity to destroy type I collagen fibers without generating bubbles in the surrounding medium. However, PAH-ICG and HSA-ICG could induce VNB generation after a single pulse at an energy of 4.5 J/cm² (FIG. 3B). In the case of LIP-ICG, no VNBs were observed for all tested light intensities, highly likely because a part of the ICG is free in the aqueous core (FIG. 3B).

ICG can Efficiently Destroy Human Vitreous Opacities Obtained after Vitrectomy

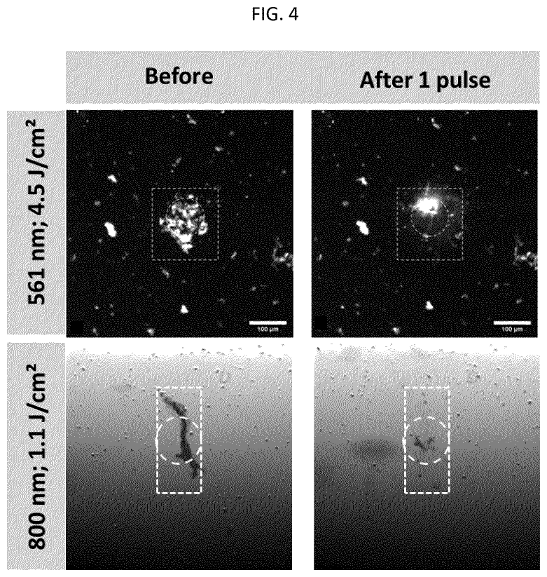

Because opacities in the eye differ in composition and consist in a mixture of different types of collagen, it was tested whether ICG could break such opacities in a similar manner as for type I collagen fibers. Vitreous containing opacities obtained from patients with eye floaters were therefore mixed with ICG (0.5 mg/ml) and illuminated with the nanosecond laser at 561 nm (4.5 J/cm²) and 800 nm (1.1 J/cm²), respectively. At both wavelengths, it appears clearly that the vitreous opacity could be destroyed as shown in FIG. 4.

VNBs Generated from ICG Destroyed Collagen Fibers In Vivo

Next, it was studied to which extent the combined use of laser pulses and ICG could destroy collagen fibers intravitreally (IVIT) injected in the eyes of rabbits. In a first series of experiments, ICG-labeled collagen fibers were prepared by mixing collagen fibers and free ICG (1.25 mg/mL in water). Then, the ICG-fibers were injected in the eyes of rabbits (FIG. 5, upper panel). The first observation was that the IVIT injected ICG-labeled fibers could be easily imaged by PAM at 578 and 800 nm. At λ 578 nm, both blood vessels and ICG-fibers could be detected while at λ 800 nm, only ICG-fibers could be observed (FIG. 5, lower panel).

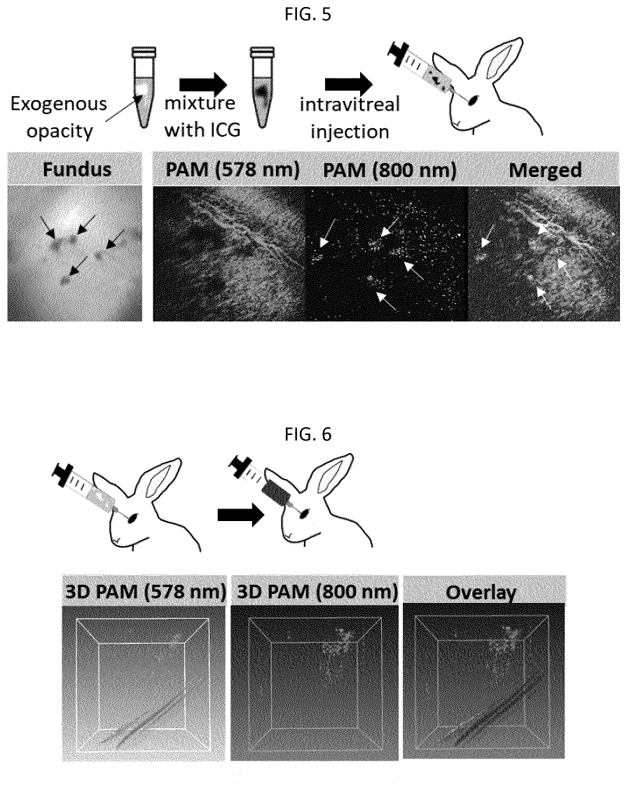

In a next series of experiments, IVIT (non-labeled) collagen fibers were injected and, 5 days later, ICG (1.25 mg/mL) (FIG. 6, upper panel). While the collagen fibers could be observed by OCT, PAM-imaging revealed that ICG co-localized with the fibers. These results confirmed that IVIT injected ICG could reach and bind the collagen fibers (FIG. 6, lower panel).

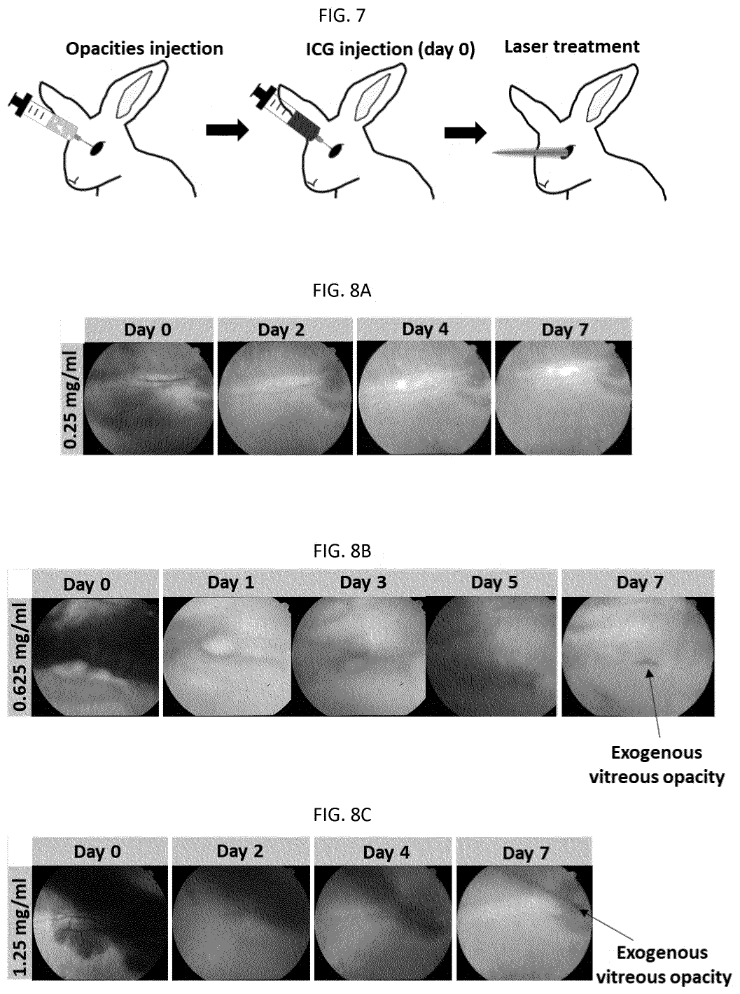

In a further experiment, as illustrated in FIG. 7, IVIT (non-labeled) collagen fibers were injected and, 5 days later, ICG (day 0). Subsequently, the collagen fibers were irradiated with laser pulses 3 days after ICG injections (day 3).

As shown in FIG. 8, after intravitreal injection, ICG could be visualized by fundus imaging at all ICG concentrations (0.25, 0.625 and 1.25 mg/ml). After one day, we observed that most of the injected ICG was no longer visible, highly likely due to clearance from the vitreous. When the ICG concentration was 0.25 mg/ml, ICG was no longer visible at the level of the collagen fibers 7 days after ICG injection (FIG. 8A). Interestingly, when a sufficiently high ICG concentration was injected (i.e. 0.625 and 1.25 mg/ml), after 7 days ICG remained only visible at the level of the collagen fibers (FIGS. 8B and 8C).

Hence, following intravitreal injection of ICG, ICG binding to collagen fibers was confirmed in vivo; binding of ICG to collagen fibers could still be observed at a concentration as low as 0.625 mg/mL (FIG. 8B), which is even lower than what is used for ILM peeling (>1 mg/mL). This is attractive since the use of a lower ICG concentration will reduce its phototoxicity. Important as well, at all concentrations of intravitreally injected ICG, most of the ‘free’ ICG (i.e. not bound to collagen) was no longer visible in the vitreous cavity 3 days post injection (FIG. 8A-C).

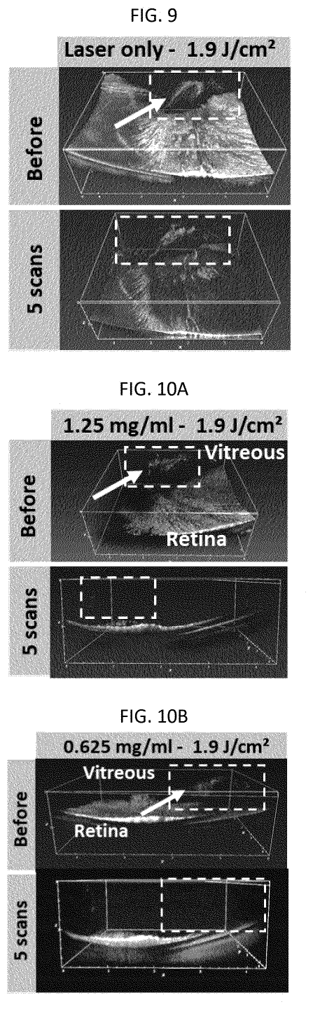

Subsequently, the collagen fibers were irradiated with laser pulses 3 days after ICG injection (day 3). As shown in FIG. 9, applying laser pulses without the use of ICG did not destroy the fibers. However, following IVIT injection of ICG (1.25 mg/mL), 5 laser pulses (i.e., 5 scans; 1.9 J/cm²) were sufficient to completely destroy a population of injected collagen fibers (FIG. 10A). Since retinal toxicity may be observed at high ICG concentration, a lower concentration (0.625 mg/mL) was tested and again a complete destruction of the collagen fibers was observed (FIG. 10B). The experiments reveal that ICG has the capacity to destroy collagen fibers in vivo using laser settings (i.e., number of pulses (scans), laser fluence) similar to those used with HA-AuNPs and at an ICG concentration which is lower than the one clinically used today for ILM peeling (usually between 1 and 5 mg/mL). On all rabbits tested (n=3), 3.3+/−1.5 pulses seemed sufficient to destroy the IVIT injected fibers.

Safety Studies

Visual inspection confirmed that eyes treated with laser (respectively without and with ICG) showed normal corneas, eyelids, anterior chamber, conjunctiva, and transparent lens. Color fundus imaging demonstrated that there were no significant changes or injuries in the posterior segment structure i.e., no hemorrhage, retinal detachment, vascular abnormalities or pigment abnormalities in the retinal pigment epithelium layer (RPE). Also, B-scan OCT images did not indicate structural disorganization in the retinal layers, a normal retinal structural anatomy was observed post laser treatment. No retinal detachment or RPE hypertrophy was observed; after laser treatment the retinal and choroidal thicknesses were slightly different from the values obtained in rabbits before treatment (N=6, p>0.05).

### CONCLUSION

The experiments show that free ICG and free trypan blue can bind and efficiently destroy type I collagen fibers at a concentration of 0.5 mg/ml and 0.1 mg/ml, respectively which are below the concentrations used in clinics (1.25 mg/ml for ICG; 0.6 mg/ml for TB) (FIG. 2A). Besides, at those concentrations, no clear toxicity on immortalized human retinal cells (MIO-M1) could be observed (FIG. 1B). At a fluence of 4.5 J/cm², a clear destruction could be observed with less than 10 pulses. This is 1000-fold lower that the current YAG laser therapy and comparable to the settings with gold nanoparticles.

A clear advantage of ICG over TB and spherical gold nanoparticles is its wide range of absorbance. Therefore, using ICG it is possible to tune the wavelength of the laser to near infrared light. One advantage of using near infrared light is a lower interference with tissues, and hence fewer side effects. The TB concentration used in the study is far below the one used in clinics (0.01 versus 0.6 mg/ml). The ICG concentration used in the study is also below the one used in clinics (0.5 versus 1.25 mg/ml).

Moreover, when ICG was encapsulated in nanoparticles, bubbles could be formed in water after illumination whereas with free ICG the bubbles could be observed only at the level of the fiber, suggesting a targeted effect. Thereby, using free ICG reduces or even avoids damages to the vitreous structure and the surrounding ocular tissues (FIG. 2C). This phenomenon is highly likely due to accumulation of the vital dyes on the fibers which decreases the energy threshold for bubble generation.

After injection of ICG in the vitreous body of rabbits previously injected with collagen fibers, it was of high interest to observe that scanning a 4.5×4.5 mm² area in the vitreous in which the collagen fibers were located efficiently destroyed the collagen fibers in vivo (FIG. 10), which was not the case without ICG (FIG. 9). Importantly, ICG allows to destroy the fibers in vitreous at a light dose which is significantly lower than the light dose applied today in the clinic with the YAG laser. This reduction of light energy makes floater treatment much safer and opens the possibility to treat vitreous opacities located close to the retina. Besides, the capacity to ‘see’ the ICG labeled opacities in the vitreous body through PAM-imaging, allows for a more precise application of the laser irradiation, further reducing the light dose needed as for YAG laser therapy. Important as well, 30 days post treatment, rabbits showed normal corneas, eyelids, anterior chamber, conjunctiva and transparent lenses. Taken together, injecting ICG and applying laser pulses in the vitreous seems an effective and safe strategy to destroy opacities.

In conclusion, this work shows that vital dyes that are approved in ophthalmology are useful for pulsed-laser therapy of vitreous opacities responsible for the phenomenon of floaters.

1. A method of treating a vitreous opacity-related disease in a subject, the method comprising:
administering a dye to a vitreous body of an affected eye of the subject; and
irradiating at least part of the vitreous opacity, thereby inducing destruction of the vitreous opacity in the subject.

2. (canceled)

3. The method according to claim 1, wherein the dye is a vital dye; preferably wherein the dye is a vital dye approved for ophthalmological use.

4. The method according to claim 1, wherein the dye is a vital dye selected from the group consisting of: Indocyanine Green, Trypan Blue, Brilliant Blue, Janus green B, Gentian violet, Bromophenol Blue, Patent blue, Light Green, Fast Green, Infracyanine Green, Methylene blue, Toluidine blue, Fluorescein Sodium, Rose Bengal, and Rhodamine 6G; preferably wherein the dye is Indocyanine Green or Trypan Blue; more preferably wherein the dye is Indocyanine Green.

5. The method according to claim 1, wherein the dye is administered at a concentration of about 0.001 mg/ml to about 0.5 mg/ml.

6. The method according to claim 1, wherein the dye is a free dye, an aggregate of the dye, or a crystal of the dye, or wherein the dye is conjugated to an agent and/or is comprised in a particle, such as a nanoparticle or a microparticle; preferably wherein the dye is grafted on a particle and/or wherein the dye is encapsulated in a particle.

7. The method according to claim 1, wherein the dye is administered to the vitreous body by intravitreal administration; preferably wherein the dye is administered to the vitreous body by intravitreal injection.

8. The method according to claim 1, wherein the dye is capable of diffusing in the vitreous body after administration.

9. The method according to claim 1, wherein the dye is capable of binding to the vitreous opacity after administration.

10. The method according to claim 1, wherein the dye is capable of forming vapor nanobubbles at the vitreous opacity when irradiated.

11. The method according to claim 1, wherein the at least part of the vitreous opacity is irradiated with electromagnetic radiation; preferably wherein the at least part of the vitreous opacity is irradiated with laser radiation; more preferably wherein the at least part of the vitreous opacity is irradiated with pulsed-laser radiation.

12. The method according to claim 11, wherein:
the intensity of the pulses of the laser may be at least 104 W/cm²;
the number of pulses of the laser may be at least 1 laser pulse;
the pulses of the laser may have a duration in the range of at least 10 fs; and/or
the laser irradiation reaches a fluence of at least 0.1 mJ/cm² at the vitreous opacity.

13. The method according to claim 1, wherein the vitreous opacity-related disease is myodesopsia or posterior vitreous detachment.

14. A method of photodestruction of a vitreous opacity in an eye of a subject, the method comprising:
administering a dye to a vitreous body of the eye of the subject; and
irradiating at least part of the vitreous opacity, thereby inducing destruction of the vitreous opacity of the subject.

15. (canceled)

16. The method according to claim 14, wherein one or more of:
the dye is a vital dye;
the dye is a vital dye approved for ophthalmological use;
the dye is a vital dye selected from the group consisting of: Indocyanine Green, Trypan Blue, Janus green B, Gentian violet, Bromophenol Blue, Patent blue, Brilliant Blue, Light Green, Fast Green, Infracyanine Green, Methylene blue, Toluidine blue, Fluorescein Sodium, Rose Bengal, and Rhodamine 6G; preferably the dye is Indocyanine Green or Trypan Blue; more preferably the dye is Indocyanine Green;
the dye is administered at a concentration of about 0.001 mg/ml to about 0.5 mg/ml;
the dye is a free dye, an aggregate of the dye, or a crystal of the dye;
the dye is conjugated to an agent;
the dye is comprised in a particle, such as a nanoparticle or a microparticle; preferably wherein the dye is grafted on a particle and/or wherein the dye is encapsulated in a particle;
the dye is administered to the vitreous body by intravitreal administration; preferably the dye is administered to the vitreous body by intravitreal injection;
the dye is capable of diffusing in the vitreous body after administration;
the dye is capable of binding to the vitreous opacity after administration;
the dye is capable of forming vapor nanobubbles at the vitreous opacity when irradiated;
the at least part of the vitreous opacity is irradiated with electromagnetic radiation; preferably wherein the at least part of the vitreous opacity is irradiated with laser radiation; more preferably wherein the at least part of the vitreous opacity is irradiated with pulsed-laser radiation;
the intensity of the pulses of the laser may be at least 104 W/cm²;
the number of pulses of the laser may be at least 1 laser pulse;
the pulses of the laser may have a duration in the range of at least 10 fs; and/or
the laser irradiation reaches a fluence of at least 0.1 mJ/cm² at the vitreous opacity.

17-20. (canceled)
